SEQUENCE LISTING (12 sequences)

SEQ ID NO: 1 - protein, 5 aa, ARTIFICIAL SEQUENCE
  VH CDR1
SYAMS

SEQ ID NO: 2 - protein, 17 aa, ARTIFICIAL SEQUENCE
  VH CDR2
GISAIDQSTYYADSVKG

SEQ ID NO: 3 - protein, 17 aa, ARTIFICIAL SEQUENCE
  VH CDR3
QKFMQLWGGGLRYPFGY

SEQ ID NO: 4 - protein, 126 aa, ARTIFICIAL SEQUENCE
  VH
EVQLLESGGGLVQPGGSLRLSCAASGFTFSSYAMSWVRQAPGKGLEWVSGISAIDQSTYYADSVKGRFTISRDNSKNTLYLQMNSLRAEDTAVYYCARQKFMQLWGGGLRYPFGYWGQGTMVTVSS

SEQ ID NO: 5 - protein, 11 aa, ARTIFICIAL SEQUENCE
  VL CDR1
SGEGMGDKYAA

SEQ ID NO: 6 - protein, 7 aa, ARTIFICIAL SEQUENCE
  VL CDR2
RDTKRPS

SEQ ID NO: 7 - protein, 9 aa, ARTIFICIAL SEQUENCE
  VL CDR 3
GVIQDNTGV

SEQ ID NO: 8 - protein, 106 aa, ARTIFICIAL SEQUENCE
  VL
SYVLTQPPSVSVSPGQTASITCSGEGMGDKYAAWYQQKPGQSPVLVIYRDTKRPSGIPERFSGSNSGNTATLTISGTQAMDEADYYCGVIQDNTGVFGGGTKLTVL

SEQ ID NO: 9 - protein, 456 aa, ARTIFICIAL SEQUENCE
  HC
EVQLLESGGGLVQPGGSLRLSCAASGFTFSSYAMSWVRQAPGKGLEWVSGISAIDQSTYYADSVKGRFTISRDNSKNTLYLQMNSLRAEDTAVYYCARQKFMQLWGGGLRYPFGYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 212 aa, ARTIFICIAL SEQUENCE
  LC
SYVLTQPPSVSVSPGQTASITCSGEGMGDKYAAWYQQKPGQSPVLVIYRDTKRPSGIPERFSGSNSGNTATLTISGTQAMDEADYYCGVIQDNTGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - DNA, 378 bp, ARTIFICIAL SEQUENCE
  VH encoding polynucleotide
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggcatttctgcaatagaccaaagcacatactacgcagactccgtgaagggccggttcaccatctccagagacaattccaagaacacgctgtatctgcaaatgaacagcctgagagccgaggacacggccgtgtattactgtgcccggcagaagttcatgcagctatggggggggggcttgcgttatcccttcggctactggggccaggggacaatggtcaccgtctcctca

SEQ ID NO: 12 - DNA, 318 bp, ARTIFICIAL SEQUENCE
  VL encoding polynucleotide
tcctatgtgctgactcagccaccctcagtgtccgtgtccccaggacagacggccagcatcacctgctctggagaaggaatgggggataaatatgctgcctggtatcagcagaagccaggccagtcacctgtgctggtcatctatcgagatacaaagcggccctcagggatccctgagcgattctctggctccaactctgggaacacagccacgttgaccatcagcgggacccaggctatggatgaggctgactattactgtggggtgatccaggacaacactggggtattcggcggagggaccaagctcaccgtccta

(19) United States
(12) Patent Application Publication
(10) Pub. No.: US 2023/0174638 A1
(43) Pub. Date: Jun. 8, 2023

(54) FORMULATIONS OF ANTI-IL-33 ANTIBODIES

(71) Applicant: MedImmune Limited, Cambridge (GB)

(72) Inventors: Sofia EKIZOGLOU, Cambridge (GB); Mahammad Syed Mastafa AHMED, Cambridge (GB); Reza ESFANDIARY, Gaithersburg, MD (US); Arun Parupudi, Gaithersburg, MD (US)

(21) Appl. No.: 17/998,388

(22) PCT Filed: May 10, 2021
(86) PCT No.: PCT/EP2021/062310

§ 371 Date: Nov. 10, 2022

(30) Foreign Application Priority Data
May 11, 2020 (GR) 20200100239

(51) Int. Cl. C07K 16/24; A61P 37/02

(52) U.S. Cl. CPC C07K 16/244; A61P 37/02; C07K 2317/565

(57) ABSTRACT

The present disclosure is directed to compositions comprising greater than about 100 mg/ml of an anti-IL-33 antibody, a surfactant, arginine and a buffer. Methods for making the compositions and methods of treating disease in a subject are also disclosed.

The application claims priority to Greek Patent Application number 20200100239, filed 11^{th} May 2020. The contents of this application is incorporated herein by reference in its entirety.

### TECHNICAL FIELD

The disclosure relates to anti-IL-33 antibodies, including high-concentration aqueous formulations of 33_640087-7B and biosimilars thereof.

### BACKGROUND TO THE DISCLOSURE

33_640087-7B is a human immunoglobulin (Ig) G1 monoclonal antibody (mAb) that binds to human interleukin (IL)-33, prevents binding of IL-33 to its receptor ST2, and inhibits conversion to disulfide-bonded (DSB) IL 33. 33_640087-7B has therapeutic potential in numerous diseases and is currently being developed for the treatment of moderate to severe chronic obstructive pulmonary disease (COPD), asthma, and atopic dermatitis (AD); and diabetic kidney disease (DKD).

It is intended for 33_640087-7B to be administered in the future via sub-cutaneous injection. The present disclosure addresses this need.

### SUMMARY OF THE DISCLOSURE

A Phase 1 clinical study of 33_640087-7B (Study D9180C00001) has been completed. Study D9180C00001 was a first in human, randomised, placebo controlled, blinded (investigator and participant blinded; sponsor unblinded) clinical study in 88 participants (Part I: single ascending dose (SAD) in 56 healthy volunteers with a history of mild atopy; Part II: multiple ascending dose (MAD) in 24 participants with mild chronic obstructive pulmonary disease; Part III: single dose in 8 healthy Japanese volunteers) to evaluate the safety, tolerability, PK, and immunogenicity of 33_640087-7B. 33_640087-7B was found to be generally safe and well tolerated, and there were no safety concerns following administration up to 300 mg 33_640087-7B intravenous (IV) in Parts I and III of the study.

In Part 2 of the study, COPD patients received 3 doses of 33_640087-7B subcutaneously at 14-day intervals.

For the Phase 1 clinical study, 33_640087-7B was supplied vialed as a sterile, white-to-off-white, lyophilized powder. Each vial comprised a nominal 50 mg of active 33_640087-7B intended for IV or SC administration. Upon reconstitution with 1.2 mL of sterile water for injection, the solution contained 50 mg/mL 33_640087-7B in 20 mM L histidine/L histidine-hydrochloride, 80 mM L-arginine-hydrochloride, 120 mM sucrose, 0.02% (weight/volume [w/v]) polysorbate 80, pH 6.0.

The predicted efficacious dose of 33_640087-7B may be as much as 300 mg (or greater) for certain conditions. Low concentration formulations may therefore provide a barrier for subcutaneous administration, particularly for use in chronic conditions. It would be unrealistic to expect patients suffering for chronic disorders to be routinely administered 6 ml or more of drug product subcutaneously in order receive a therapeutic dose.

As such, there is a need to increase the concentration of 33_640087-7B in drug formulations, particularly for subcutaneous administration.

However, increasing the concentration of protein in drug formulations can cause problems with stability, for example protein aggregation resulting in formation of high molecular weight species (HMWS). HMWS, particularly those that conserve most of the native configuration of the antibody, can be of particular concern in some protein formulations. Soluble, higher order species may form due to reversible self-association (RSA) induced by high protein concentration. Aggregation can also potentially affect the subcutaneous bioavailability and pharmacokinetics of a therapeutic protein. In addition, the formation of soluble, higher order species at high concentrations may increase solution viscosity, making it difficult to deliver drug product, particularly from devices with high back pressure, such as pre-filled syringes.

Provided herein is an improved formulation for anti-IL-33 antibodies having low viscosity and reduced reversible self-association characteristics while containing a high concentration of antibody.

In one aspect, the disclosure provides a composition comprising greater than about 100 mg/ml of an anti-IL-33 antibody, at least about 170 mM arginine and a buffer, wherein the anti-IL-33 antibody comprises: a heavy chain variable domain comprising a VHCDR1 having the sequence of SEQ ID NO: 1, a VHCDR2 having the sequence of SEQ ID NO: 2, a VHCDR3 having the sequence of SEQ ID NO: 3; and a heavy chain variable domain comprising a VLCDR1 having the sequence of SEQ ID NO: 5, a VLCDR2 having the sequence of SEQ ID NO: 6, and a VLCDR3 having the sequence of SEQ ID NO: 7. In some instances, the composition further comprises a surfactant.

In another aspect, the disclosure provides a composition comprising greater than about 100 mg/ml of an anti-IL-33 antibody, at least about 150 mM lysine and a buffer, wherein the anti-IL-33 antibody comprises: a heavy chain variable domain comprising a VHCDR1 having the sequence of SEQ ID NO: 1, a VHCDR2 having the sequence of SEQ ID NO: 2, a VHCDR3 having the sequence of SEQ ID NO: 3; and a heavy chain variable domain comprising a VLCDR1 having the sequence of SEQ ID NO: 5, a VLCDR2 having the sequence of SEQ ID NO: 6, and a VLCDR3 having the sequence of SEQ ID NO: 7. In some instances, the composition further comprises a surfactant.

In some instances, the composition is a liquid. In some instances, the composition is characterized by having a reduced viscosity, relative to a composition comprising a lower concentration of the anti-IL-33 antibody, 20 mM histidine, 80 mM arginine, 120 mM sucrose and 0.02% (w/v) polysorbate 80 at pH 6.0. In some instances, the composition is characterized by the anti-IL-33 antibody having reduced reversible self-association relative to the reversible self-association of the anti-IL-33 antibody in a composition comprising 20 mM histidine, 80 mM arginine, 120 mM sucrose and 0.02% (w/v) polysorbate 80 at pH 6.0 and a lower concentration of the anti-IL-33 antibody.

In a further aspect, the disclosure provides a composition comprising about 130 mg/ml to about 170 mg/ml of 33_640087-7B, about 0.03% (w/v)±0.015% polysorbate 80, about 220 mM arginine, and about 16 to about 24 mM histidine buffer, wherein the pH is pH 5.5±0.5.

In a further aspect, the disclosure provides a composition comprising about 130 mg/ml to about 170 mg/ml of an anti-IL-33 antibody, about 0.03% (w/v)±0.015% polysorbate 80, about 220 mM arginine, and about 16 to about 24 mM histidine buffer, wherein the pH is pH 5.5±0.5.

In a further aspect, the disclosure provides an article of manufacture, comprising a composition disclosed herein, for example, comprising 0.5 ml to about 5 ml (e.g., 1 ml to about 3 ml) of the composition.

In a further aspect, the disclosure provides a vial comprising a composition disclosed herein, for example, comprising about 0.5 ml to about 5 ml (e.g., 1 ml to about 3 ml) of the composition.

In a further aspect, the disclosure provides a method of treating an IL-33-mediated disorder in a subject comprising administering to the subject a therapeutically effective amount of a composition disclosed herein.

In a further aspect, the disclosure provides a method of making a stable, liquid composition having a viscosity of less than about 10 cP and comprising greater than about 100 mg/ml of an anti-IL-33 antibody, at least about 170 mM arginine, optionally a surfactant, and a buffer. The method comprises the steps of (i) combining a first solution comprising the antibody at a first concentration and a buffer, with arginine to obtain a solution comprising about 110 mg/mL to about 200 mg/mL anti-IL-33 antibody, at least about 170 mM of arginine and buffer; and, optionally (ii) adding a surfactant to the solution to achieve a final concentration of about 0.03% (w/v)±0.015% (w/v) surfactant.

In a further aspect, the disclosure provides a method of making a stable, liquid composition having a viscosity of less than about 10 cP and comprising greater than about 100 mg/ml of an anti-IL-33 antibody, at least about 150 mM lysine, optionally a surfactant, and a buffer. The method comprises the steps of (i) combining a first solution comprising the antibody at a first concentration and a buffer, with arginine to obtain a solution comprising about 110 mg/mL to about 200 mg/mL anti-IL-33 antibody, at least about 150 mM of lysine and buffer; and optionally (ii) adding a surfactant to the solution to achieve a final concentration of about 0.02% (w/v)±0.015% (w/v) surfactant.

### BRIEF DESCRIPTION OF THE DRAWINGS

Embodiments of the invention will be described, by way of example, with reference to the following drawings, in which:

FIG. 1 shows the stability of the % distribution of soluble, higher order species of 33_640087-7B a composition of the disclosure, when stored between 2° C. and 8° C. over a period of 6 months.

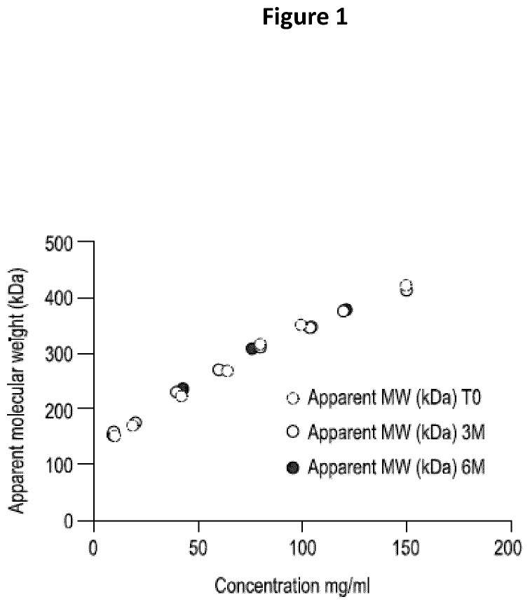

FIG. 2 shows the long-term stability of 33_640087-7B in a composition of the disclosure, measured as a function of the formation of insoluble aggregates over time (months—M). Aggregation levels were measured during storage in glass vials and pre-filled syringes (PFS). Compositions were stored between 2 and 8° C.

FIG. 3 shows the stability of 33_640087-7B in a composition of the disclosure, measured as a function of the formation of insoluble aggregates over time (months—M). Aggregation levels were measured during storage in glass vials and pre-filled syringe (PFS). Compositions were stored at 25° C.

FIG. 4 shows the stability of 33_640087-7B in a composition of the disclosure, measured as a function of the formation of insoluble aggregates over time (months—M). Aggregation levels were measured during storage in glass vials and pre-filled syringe (PFS). Compositions were stored at 40° C.

FIG. 5 shows the impact of arginine and temperature on formulation viscosity.

FIG. 6 shows the impact of 33-640087_7B concentration, arginine and temperature on formulation viscosity. Viscosity (Cp) was measured at each 33-640087_7B concentration at each arginine concentration at 5° C., 18° C., 25° C. and 30° C.

### DETAILED DESCRIPTION

### Definitions

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference.

As used in this specification, the singular forms “a,” “an” and “the” specifically also encompass the plural forms of the terms to which they refer, unless the content clearly dictates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

The practice of a method disclosed herein, and individual steps thereof, can be performed manually and/or with the aid of or automation provided by electronic equipment. Although processes have been described with reference to particular instances, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

The compositions and methods are contemplated to include embodiments including any combination of one or more of the additional optional elements, features, and steps further described below (including those shown in the figures), unless stated otherwise.

Unless otherwise defined, all technical and scientific terms used herein have the same meaning as commonly understood by one of ordinary skill in the art to which the present disclosure belongs. The following references provide one of skill with a general definition of many of the terms used in this disclosure include, but are not limited to: Singleton et al., DICTIONARY OF MICROBIOLOGY AND MOLECULAR BIOLOGY (2d Ed. 1994); THE CAMBRIDGE DICTIONARY OF SCIENCE AND TECHNOLOGY (Walker Ed., 1988); THE GLOSSARY OF GENETICS, 5th Ed., R. Rieger et al. (Eds.), Springer Verlag (1991); and Hale & Marham, THE HARPER COLLINS DICTIONARY OF BIOLOGY (1991).

The term “antibody” or “immunoglobulin” refers to a tetrameric glycoprotein that consists of two heavy chains and two light chains, each comprising a variable region and a constant region. Antigen-binding portions may be produced by recombinant DNA techniques or by enzymatic or chemical cleavage of intact antibodies. The term “antibody” includes monoclonal antibodies, polyclonal antibodies, chimeric antibodies, human antibodies, and humanized antibodies.

Antibody variants include antibody fragments and anti-body like proteins with changes to structure of canonical tetrameric antibodies. Typically antibody variants include V regions with a change to the constant regions, or, alternatively, adding V regions to constant regions, optionally in a non-canonical way. Examples include multispecific antibodies (e.g., bispecific antibodies with extra V regions), antibody fragments that can bind an antigen (e.g., Fab′, F′(ab)2, Fv, single chain antibodies, diabodies), biparatopic and recombinant peptides comprising the forgoing as long as they exhibit the desired biological activity.

Antibody fragments include antigen-binding portions (i.e., antigen-binding fragments”) of the antibody including, inter alia, Fab, Fab′, F(ab′)2, Fv, domain antibody (dAb), complementarity determining region (CDR) fragments, CDR-grafted antibodies, single-chain antibodies (scFv), single chain antibody fragments, chimeric antibodies, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

The terms “treat”, “treating” and “treatment” refer to eliminating, reducing, suppressing or ameliorating, either temporarily or permanently, either partially or completely, a clinical symptom, manifestation or progression of an event, disease or condition associated with a disorder described herein. As is recognized in the pertinent field, drugs employed as therapeutic agents may reduce the severity of a given disease state but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the disclosure is directed to a method for determining the efficacy of treatment comprising administering to a patient therapeutic agent in an amount and for a time sufficient to induce a sustained improvement over baseline of an indicator that reflects the severity of the particular disorder.

‘IL-33’ protein as employed herein refers to interleukin 33, in particular a mammalian interleukin 33 protein, for example human protein deposited with UniProt number 095760. IL-33 exists in reduced and oxidized forms. The terms “IL-33” and “IL-33 polypeptide” are used interchangeably. In certain embodiments, IL-33 is full length. In another embodiment, IL-33 is mature, truncated IL-33 (amino acids 112-270). Recent studies suggest full length IL-33 is active (Cayrol and Girard, Proc Natl Acad Sci USA 106(22): 9021-6 (2009); Hayakawa et al., Biochem Biophys Res Commun. 387(1):218-22 (2009); Talabot-Ayer et al, J Biol Chem. 284(29): 19420-6 (2009)). However, N-terminally processed or truncated IL-33 including but not limited to aa 72-270, 79-270, 95-270, 99-270, 107-270, 109-270, 111-270, 112-270 may have enhanced activity (Lefrancais 2012, 2014).

‘Oxidized IL-33’ or ‘oxIL-33’ as employed herein refers to the form of the IL-33 that binds to RAGE, and triggers RAGE mediated signalling. Oxidised IL-33 is a protein visible as a distinct band, for example by western blot analysis under non-reducing conditions, in particular with a mass 4 Da less than the corresponding reduced from. In particular, it refers to a protein with one or two disulphide bonds between the cysteines independently selected from cysteines 208, 227, 232 and 259. In one embodiment, oxidized IL-33 shows no binding to ST2.

‘Reduced IL-33’ or ‘redIL-33’ as employed herein refers to the form of the IL-33 that binds to ST2 and triggers ST2 mediated signalling. In particular cysteines 208, 227, 232 and 259 of the reduced form are not disulfide bonded. In one embodiment, reduced IL-33 shows no binding to RAGE.

The term “IL-33-mediated disorder,” as used herein, refers to any disorder or condition mediated by, or associated with, the IL-33 axis. In some instances, IL-33-mediated disorders are associated with excess IL-33 levels or activity in which atypical symptoms may manifest due to the levels or activity of IL-33 locally and/or systemically in the body. Exemplary IL-33-mediated disorders include inflammatory conditions, immune disorders, fibrotic disorders, eosinophilic disorders, infections, pain, central nervous system disorders, solid tumors, and ophthalmologic disorders. IL-33-mediated disorders are described, for example, in Liew et al. Nature Reviews Immunology 10: 103-110, 2010, which is incorporated herein by reference in its entirety. An “IL33-mediated disorder” may also herein be referred to a “IL33-driven disorder”.

In some instances, the IL-33-mediated inflammatory disease may be any of asthma, sepsis, septic shock, atopic dermatitis, allergic rhinitis, rheumatoid arthritis, chronic obstructive pulmonary disease (COPD), asthma, COPD overlap syndrome (ACOS), chronic bronchitis, emphysema, chronic rhinosinusitis with or without nasal polyps, vasculitis, GvHD, uveitis, chronic idiopathic urticaria, sinusitis or pancreatitis.

In some instances, the IL-33-mediated disorder may be diabetic kidney disease. Diabetic kidney disease defined herein refers to a diagnosis of Type II Diabetes Mellitus and an estimated glomerular filtration rate (eGFR) of 30-75 ml/min. Typically, DKD is further defined as a diagnosis of UACR ratio of from 100 to 3000 mg albumin to g creatinine. The term “therapeutically effective amount” refers to an amount of therapeutic agent that is effective to ameliorate or lessen symptoms or signs of disease associated with a disease or disorder.

Compositions with Low Reversible Self-Association

33_640087-7B has been shown to be safe and generally well-tolerated in humans in doses up to 300 mg administered parenterally. Depending on the disease and the local biology of interleukin-33, a therapeutically effective amount of 33_640087-7B may be equal to or more than 300 mg.

Many conditions in which IL-33 is thought to mediate disease are chronic and long-lasting. That means that for a patient to effectively manage symptoms of disease they may need to be chronically administered anti-IL-33-based therapies. Therefore, a composition comprising 33_640087-7B that is suitable for therapeutic use should make prolonged treatment as comfortable as possible for a patient.

As such, high concentration formulations suitable for parenteral administration (e.g., subcutaneous or intravenous administration) are desired. However, formulations with high protein concentrations can be challenging from the standpoint of developability. For example, 33_640087-7B has been shown to reversibly self-associate at moderately high concentrations (e.g., at about 50 mg/ml). Reversible self-association (RSA) is an important developability parameter for high concentration formulations. RSA often manifests in the form of soluble, reversible, higher order species, (e.g. non-covalent dimers) and can introduce challenges during manufacturing and drug administration. For example, presence of RSA can lead to an increase in viscosity. High viscosity can present significant manufacturing challenges, for example, by blocking filters during the filtration processes. This in turn can lead to a reduction in yield if a significant amount of drug substance is lost when higher order species are purified from monomer during the purification process. Increased viscosity may also negatively impact drug administration by reducing the functionality of the device used to administer the antibody, particularly where the drug is administered parenterally, such as by sub-cutaneous administration. In such circumstances. the ability of the end user (e.g., a patient or a health care provider) to manually inject the may be compromised by high viscosity.

Provided herein are stable, liquid compositions (i.e. “liquid formulations”) suitable for parenteral administration that may be stored long term or short term comprising a high concentration of an anti-IL-33 antibody (meaning greater than 100 mg/ml), optionally a surfactant, a high concentration of arginine or lysine, and a buffer. As used herein, “high concentration of arginine” refers to a concentration of arginine greater than about 170 mM, such as greater than about 190 mM. A “high concentration of lysine” refers to a concentration of lysine greater than about 150 mM. The composition disclosed herein has been shown to surprisingly reduce the reversible self-association (RSA) of 33_640087-7B in high concentration liquid compositions relative to the RSA previously observed at lower 33_640087-7B concentrations in the Phase I formulation (see Example 1).

In certain instances, the anti-IL-33 antibody is present in the composition at a concentration greater than about 100 mg/ml, and optionally less than about 200 mg/ml. In some instances, the anti-IL-33 antibody is present in the composition at a concentration of about 105 mg/ml, about 110 mg/ml, about 115 mg/ml, about 120 mg/ml, about 125 mg/ml, about 130 mg/ml, about 135 mg/ml, about 140 mg/ml, about 145 mg/ml, about 150 mg/ml, about 155 mg/ml, about 160 mg/ml, about 165 mg/ml, about 170 mg/ml, about 175 mg/ml, about 180 mg/ml, about 190 mg/ml, or about 195 mg/ml. In some instances, the anti-IL-33 antibody is present in the composition at a concentration of about 105 mg/ml to about 190 mg/ml, about 110 mg/ml to about 180 mg/ml, about 110 mg/ml to about 170 mg/ml, about 110 mg/ml to about 165 mg/ml, about 110 mg/ml to about 160 mg/ml, about 120 mg/ml to about 160 mg/ml, about 130 mg/ml to about 160 mg/ml, or 140 mg/ml to about 160 mg/ml. In some instances, the anti-IL-33 antibody is present in the composition at a concentration of about 110 mg/ml±10%, about 115 mg/ml±10%, about 120 mg/ml±10%, about 125 mg/ml±10%, about 130 mg/ml±10%, about 135 mg/ml±10%, about 140 mg/ml±10%, about 145 mg/ml±10%, about 150 mg/ml±10%, about 155 mg/ml±10%, about 160 mg/ml±10%, about 165 mg/ml±10%, about 170 mg/ml±10%, about 175 mg/ml±10%, about 180 mg/ml±10%, about 185 mg/ml±10%, about 190 mg/ml±10% or about 195 mg/ml±10%. In some instances, the anti-IL-33 antibody is present in the compositions at a concentration of about 150 mg/ml.

In some instances, the compositions of the present disclosure comprise a surfactant. Surfactants are surface active agents that are amphipathic (having a polar head and hydrophobic tail). Surfactants preferentially accumulate at interfaces, resulting in reduced interfacial tension. Use of a surfactant can also help to mitigate formation of large proteinaceous particles. In some instances, the surfactant present in the compositions of the present disclosure is an amphipathic and/or nonionic surfactant. Exemplary surfactants include polyoxyethylene sorbitan fatty acidesters (e.g. polysorbate 20, polysorbate 80), alkylaryl polyethers, e.g. oxyethylated alkyl phenol (e.g. Triton™ X-100), and poloxamers (e.g. Pluronics®, e.g. Pluronic® F68), and combinations of any of the foregoing, either within a class of surfactants or among classes of surfactants. Polysorbate 20 and polysorbate 80 (and optionally mixtures thereof) are particularly contemplated. The surfactant in exemplary instances is present in the composition at a concentration of less than or about 0.050% (w/v)±0.015% (w/v). For instance, the composition may comprise about 0.005% (w/v) to about 0.05% (w/v) surfactant, e.g., about 0.005% (w/v), about 0.015% (w/v), about 0.02% (w/v), about 0.025% (w/v), about 0.03% (w/v), about 0.035% (w/v), about 0.04% (w/v), about 0.045% (w/v), or about 0.05% (w/v). In some instances, the surfactant in the composition is at a concentration of 0.03% (w/v)±0.015% (w/v). In some instances, the surfactant in the composition is at a concentration of 0.03% (w/v)±0.01% (w/v). In some instances, the surfactant in the composition is at a concentration of about 0.02% (w/v) to about 0.04% (w/v). In some instances, the surfactant comprises polysorbate 80.

The composition of the present disclosure also comprises at least about 170 mM arginine. In some instances, the composition comprises at least about 190 mM arginine. In some instances, the compositions of the present disclosure comprise L-arginine. In some instances, the compositions of the present disclosure comprise L-arginine-hydrochloride. In some instances, the composition comprises greater than 190 mM arginine. In some instances, the composition comprises less than about 500 mM arginine. For example, the composition comprises about 200 mM, about 210 mM, about 220 mM, about 240 mM, about 260 mM, about 280 mM, about 300 mM, about 350 mM, about 400 mM, or about 450 mM arginine. In some instances, the composition comprises about 220 mM arginine. It has been found that a high concentration of arginine (i.e. a concentration of greater than about 190 mM) contributes to a surprising reduction in the reversible self-association of 33_640087-7B in a stable, liquid composition, relative to the reversible self-association observed in a composition comprising 20 mM histidine, 80 mM arginine, 120 mM sucrose, 0.02% (w/v) polysorbate 80 at pH 6.0 and a lower concentration of 33_640087-7B (about 50 mg/ml). In some instances, the composition of the present disclosure comprises about 220 mM arginine when the composition comprises about 150 mg/ml of an anti-IL-33 antibody.

In another aspect, the composition of the present disclosure alternatively comprises at least about 150 mM of lysine. In some instances, the compositions of the present disclosure comprise L-lysine. In some instances, the compositions of the present disclosure comprise L-lysine-hydrochloride. In some instances, the composition comprises greater than 150 mM lysine. In some instances, the composition comprises less than about 500 mM lysine. For example, the composition comprises about 160 mM, about 170 mM, about 180 mM, about 200 mM, about 220 mM, about 240 mM, about 260 mM, about 280 mM, about 300 mM, about 350 mM, about 400 mM, or about 450 mM lysine. In some instances, the composition comprises about 170 mM lysine. It has been found that a high concentration of lysine (i.e. a concentration of greater than about 150 mM) contributes to a surprising reduction in the viscosity of 33_640087-7B in a stable, liquid composition, relative to the viscosity observed in a composition comprising 20 mM histidine, 80 mM arginine, 120 mM sucrose, 0.02% (w/v) polysorbate 80 at pH 6.0 and a lower concentration of 33_640087-7B (about 50 mg/ml). In some instances, the composition of the present disclosure comprises about 170 mM lysine when the composition comprises about 150 mg/ml of an anti-IL-33 antibody.

In some instances, the composition of the present disclosure is characterized by the anti-IL-33 antibody having reduced reversible self-association relative to a composition comprising 20 mM histidine, 80 mM arginine, 120 mM sucrose, 0.02% (w/v) polysorbate 80 at pH 6.0 and a lower concentration of the anti-IL-33 antibody. In some instances, the RSA is reduced 2-fold relative to the RSA of the anti-IL-33 antibody in a liquid composition comprising the lower concentration of the anti-IL-33 antibody in 20 mM histidine, 80 mM arginine, 120 mM sucrose, 0.02% (w/v) polysorbate 80 at pH 6.0. In some instances, the lower concentration of anti-IL-33 antibody is 50 mg/ml. RSA positively correlates with protein concentration. Therefore, it would be expected that increasing the concentration of the therapeutic protein would lead to an increase in RSA of the therapeutic protein. RSA can be expressed as a function of the % monomer of the soluble species in the liquid composition. The examples unexpectedly show that a novel formulation can increase the weight % monomer 2-fold when the concentration of the therapeutic protein is increased 3-fold, relative to the weight % monomer observed in a composition comprising 20 mM histidine, 80 mM arginine, 120 mM sucrose, 0.02% (w/v) polysorbate 80 at pH 6.0. In some instances, the composition of the disclosure comprises a weight % monomer of at least about 30%, for example, about 30%, about 31%, about 32%, about 33%, about 34%, about 35%, about 36%, about 37%, about 38%, about 39%, about 40%, about 41% about 42%, about 43%, about 44%, about 45%, about 46%, about 47%, about 48%, about 49% or about 50%. In some instances, the composition of the disclosure comprises a weight % monomer of from about 35% to about 50%. In some instances, the composition of the disclosure comprises a weight % monomer of about 40% to about 45%. In some instances, the composition of the disclosure comprises a weight % monomer of from about 35% to about 50% after storage for about 3 months at a temperature of from about 2° C. to about 8° C. In some instances, the composition of the disclosure comprises a weight % monomer of from about 40% after storage for about 3 months at a temperature of from about 2° C. to about 8° C. In some instances, the composition of the disclosure comprises a weight % monomer of from about 35% to about 50% after storage for about 6 months at a temperature of from about 2° C. to about 8° C. In some instances, the composition of the disclosure comprises a weight % monomer of from about 40% after storage for about 6 months at a temperature from about 2° C. to about 8° C. RSA and % monomer can be calculated using static light scattering intensity in volts measured as a function of concentration (mg/mL). It may then be converted into apparent molecular weight (kDa) at each concentration using the Rayleigh equation. Unless otherwise stated, this method has been used to measure RSA in the stable, liquid compositions disclosed herein.

The composition of the present disclosure comprises a buffer. The buffer can be, for instance, an organic buffer. In some instances, the buffer is centered at 25° C. around pH 5 to pH 6.5, or to pH 5 to pH 6. In some embodiments, the buffer can have a pKa within one pH unit of pH 5.4 to pH 5.6 at 25° C. An exemplary buffer is histidine/histidine hydrochloride, which has a pKa of about pH 6.09 at 25° C. Another such buffer is acetic acid/acetate, having a pKa of about 4.75 at 25° C. Other alternative buffers contemplated include buffers based on ions including propionate (pKa of 4.87 at 25° C.), malate (pKa of 5.13 at 25° C.), pyridine (pKa of 5.23 at 25° C.), piperazine (pKa of 5.33 at 25° C.), and succinate (pKa of 5.40 at 25° C.). Buffers based on histidine are particularly contemplated. In some instances, the buffer is histidine.

In some instances, the buffer in the composition is present at a concentration of about 1 mM to about 50 mM, about 1 to 40 mM, or about 1 mM to about 30 mM. In some instances, the composition comprises about 5 mM to about 50 mM, about 10 mM to about 40 mM, about 15 to about 30 mM buffer, or about 15 to about 25 mM buffer. In some instances, the buffer in the composition is present at a concentration of about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM about 25 mM, about 26 mM, about 27 mM, about 28 mM, about 29 mM or about 30 mM. In some instances, the buffer is at a concentration of about 16 mM to about 24 mM, about 17 mM to about 24 mM, about 18 mM to about 24 mM or about 19 mM to about 21 mM. In some instances, the composition comprises about 20 mM±10% buffer.

In instances, the composition of the present disclosure may comprise additional components. The composition, in various aspects, comprises any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, UK, 2000), which is incorporated by reference in its entirety; Remington's Pharmaceutical Sciences, Sixteenth Edition, E. W. Martin (Mack Publishing Co., Easton, Pa., 1980), which is incorporated by reference in its entirety.

In some instances, the composition of the present disclosure is a liquid. In certain instances, the liquid has a pH which is less than about 6.0, optionally, about 5.5. In some instances, the pH is about 5.0 to about 6.0 or about 5.3 to about 5.8, e.g., about 5.3, about 5.4, about 5.5, about 5.6, about 5.7, or about 5.8, about 5.4. In some instances, the pH is about 5.5.

In some instances, the composition is characterized by a reduced viscosity, relative to a formulation comprising a lower concentration of the anti-IL-33 antibody, 20 mM histidine, 80 mM arginine, 120 mM sucrose, 0.02% (w/v) polysorbate 80 at pH 6.0. In some instances, the composition is characterized by a viscosity of less than about 25 centiPoise (cP) at 23° C. when the concentration of the anti-IL-33 antibody is less than 165 mg/mL, optionally about 9 cP when the concentration of the anti-Il-33 antibody is about 150 mg/mL, or about 5 cP when the concentration of the anti-IL-33 antibody is about 130 mg/mL. In certain aspects, the composition is characterized by a viscosity of about 5 cP to about 20 cP, e.g., about 5 cP to about 15 cP, about 5 cP to about 10 cP, about 10 cP to about 20 cP, about 15 cP to about 20 cP, or about 5 cP, about 6 cP, about 7 cP, about 8 cP, about 9 cP, about 10 cP, about 11 cP, about 12 cP, about 13 cP, about 14 cP, about 15 cP, about 16 cP, about 17 cP, about 18 cP, about 19 cP, about 20 cP, when the concentration of the anti-IL-33 antibody is less than about 150 mg/mL (e.g., about 130 mg/mL, about 140 mg/mL, about 150 mg/mL). In exemplary aspects, the composition has a viscosity that is about 10 cP±5 cP when the concentration of the antibody is about 130 mg/mL to about 170 mg/mL. In some instances, the composition is characterized by a viscosity of less than about 10 centiPoise (cP) at 23° C. when the concentration of the anti-IL-33 antibody is about 150 mg/mL. In some instances, the viscosity is from about 5 cP to about 20 cP, optionally less than about 10 cP, such as about 9 cP. Unless noted otherwise, all viscosities disclosed herein refers to a viscosity measured using a rotational viscometer at 23° C. and at a shear rate of about 1000 l/s.

In exemplary aspects, the composition is intended for subcutaneous administration to a subject, and thus the composition is isotonic with the intended site of administration. For example, the osmolality of the composition is, in some instances, in a range of about 340 to about 520 mOsm/kg, or about 344 to about 516 mOsm/kg, or about 400 to about 500 mOsm/kg. In exemplary instances, the liquid pharmaceutical composition has an osmolality in a range of about 300 mOsm/kg to about 600 mOsm/kg, or about 340 mOsm/kg to about 520 mOsm/kg, or about 360 mOsm/kg to about 500 mOsm/kg. In some instances, the osmolality is about 452 mOsm/kg.

The composition of the present disclosure is advantageously suitable for long-term or short-term storage. In some instances, the composition is suitable for long- or short-term storage at frozen or refrigerated temperatures or at higher temperatures. Accordingly, the compositions of the present disclosure may be stored at temperatures below 0° C. (e.g., about −80° C. to about −10° C., about −60° C. to about −20° C., or about −30° C.) or at temperatures of about 1° C. to about 10° C. (e.g., about 2° C. to about 8° C.). Optionally, the storage at these temperatures (below 10° C.) may be a long-term storage, e.g., at least 6 months, at least 12 months, at least 18 months, at least 24 months, at least 30 months, at least 36 months. The compositions of the present disclosure may be stored at room temperature (e.g., about 20° C. to about 30° C., about 23° C. to about 27° C., about 25° C., or about 30° C.). In some instances, the compositions of the present disclosure may be stored at temperatures above room temperature (e.g., greater than 30° C. (e.g., about 35° C. to about 45° C., about 40° C.).

In some instances, the composition of the present disclosure is highly stable and can endure long term storage at refrigerated or frozen temperatures. The composition of the present disclosure is highly stable as a liquid or as a solid. Optionally, less than about 5% (e.g., less than about 4%, less than about 3%, less than about 2%, less than about 1%) of the anti-IL-33 antibody aggregates after about 1 month to about 3 months of storage at about 2° C. to about 8° C. (e.g., about 2° C., about 4° C., about 8° C., about −20° C.). As used herein “aggregates” refers to insoluble aggregates of the anti-IL-33 antibody. In some instances, less than about 5% (e.g., less than about 4%, less than about 3%, less than about 2%, less than about 1%) of the anti-IL-33 antibody aggregates after 6 months or 12 months of storage at about at about 2° C. to about 8° C. (e.g., about 2° C., about 4° C., about 8° C., about 10° C.) as determined by SEC.

In some instances, less than about 5% (e.g., less than about 4%, less than about 3%, less than about 2%, less than about 1%) of the anti-IL-33 antibody is degraded after 18 months of storage at about 2° C. to about 8° C., (e.g., about 2° C., about 4° C., about 8° C.) as determined by SEC. In some instances, more than 95% of the anti-IL-33 antibody is intact after 18 months of storage at about 2° C. to about 8° C. (e.g., about 2° C., about 4° C., about 8° C.) in glass vials or syringes, as determined by SEC. In some instances, less than about 5% (e.g., less than about 4%, less than about 3%, less than about 2%, less than about 1%) of the antibody in the composition of the present disclosure aggregates after about 18 months of storage at about at about 2° C. to about 8° C., (e.g., about 2° C., about 4° C., about 8° C.) as determined by SEC.

In some instances, the composition of the present disclosure is highly stable and can endure long term storage at room temperatures. Optionally, less than about 5% (e.g., less than about 4%, less than about 3%, less than about 2%, less than about 1%) of the anti-IL-33 antibody aggregates after about 1 month to about 3 months of storage at about 23° C. to about 27° C., (e.g., about 23° C., about 24° C., about 25° C., about 26° C., about 27° C.). In some instances, less than about 5% (e.g., less than about 4%, less than about 3%, less than about 2%, less than about 1%) of the anti-IL-33 antibody aggregates after about 6 months of storage at about 23° C. to about 27° C. as determined by SEC. In instances, more than 95% of the anti-IL-33 antibody is intact after about 6 months of storage at about 23° C. to about 27° C. in glass vials or syringes, as determined by SEC In some instances, less than about 5% (e.g., less than about 4%, less than about 3%, less than about 2%, less than about 1%) of the anti-IL-33 antibody in the composition of the present disclosure is degraded after about 6 months of storage at about 23° C. to about 27° C. as determined by SEC.

In various instances, the composition of the present disclosure is highly stable and highly stable and can endure short term storage under stressed storage conditions. In some instances, less than about 5% (e.g., less than about 4%, less than about 3%, less than about 2%, less than about 1%) of the anti-IL-33 antibody aggregates after about 1 month to about 3 months of storage at about at about 38° C. to about 42° C., (e.g., about 38° C., about 39° C., about 40° C., about 41° C., about 42° C.). In some instances, more than 95% of the anti-IL-33 antibody is intact after about 3 months of storage at about at about 38° C. to about 42° C., (e.g., about 38° C., about 39° C., about 40° C., about 41° C., about 42° C.) in glass vials or syringes, as determined by SEC. In some instances, less than about 5% (e.g., less than about 4%, less than about 3%, less than about 2%, less than about 1%) of the anti-IL-33 antibody in the composition of the present disclosure aggregates after about 3 months of storage at about at about 38° C. to about 42° C., (e.g., about 38° C., about 39° C., about 40° C., about 41° C., about 42° C.), as determined by SEC.

In certain instances, the composition is provided for storage or use, e.g. in a single-use vial, single-use syringe, or glass, glass-lined, or glass-coated primary container. In exemplary instances, the composition is provided in a single use system bag or a polycarbonate carboy for frozen storage. In alternative instances, the composition is contained in glass vials or syringes for storage, e.g., long-term storage, at about 2° C. to about 8° C. or storage at higher temperatures (e.g., about 25° C., about 30° C., about 40° C.).

In certain instances, the composition is provided for use in a delivery system which is off-the-shelf and/or designed for self-administration. In certain instances, the composition is provided in a pre-filled syringe or an autoinjector, a pen injector, a dual-chamber pen, and the like. Such products are known in the art and are commercially available. See, e.g., Shire, Steven, Monoclonal Antibodies: Meeting the Challenges in Manufacturing, Formulation, Delivery and Stability of Final Drug Product, Chapter 8: Development of delivery device technology to deal with the challenges of highly viscous mAb formulations at high concentration, Woodhead Publishing, Cambridge, UK, pages 153-162 (2015).

The composition of the present disclosure can be suitable for administration by any acceptable route, including parenteral, and specifically subcutaneous. For example, the subcutaneous administration can be to the upper arm, upper thigh, or abdomen. Other routes include intravenous, intradermal, intramuscular, intraperitoneal, intranodal and intrasplenic, for example. The subcutaneous route is preferred. In some instances, the intravenous route is preferred. For example, the stable, liquid composition may be diluted in IV fluid prior to delivery via the intravenous route.

The composition disclosed herein comprises an anti-IL-33 antibody. Interleukin-33 (IL-33), also called IL-1F11, is a member of the IL-1 family of cytokines that stimulates the generation of cells, cytokines, and immunoglobulins characteristic of a type two immune response. IL-33 is a 270 amino acid protein, consisting of two domains: a homeodomain and a cytokine (IL-1-like) domain. The homeodomain contains a nuclear localization signal (NLS). IL-33 mediates signal transduction through ST2, a receptor expressed on Th2 cells, mast cells and a wide variety of other cell types.

The extracellular form of IL-33 stimulates target cells by binding to ST2 and subsequently activates NFKB and MAP Kinase pathways, leading to a range of functional responses including production of cytokines and chemokines. Soluble ST2 (sST2) is thought to be a decoy receptor, preventing IL-33 signaling.

In humans, IL-33 was found to be expressed constitutively in smooth muscle and in bronchial epithelia. Expression can be induced by IL-Iβ and TNF-a in lung and dermal fibroblasts (Schmitz et al. (2005)). The levels of soluble ST2 protein and IL-33 mRNA/protein are increased in sera and tissues from patients with asthma (Oboki et al., Allergology International 59: 143-160 (2010)).

In vivo, IL-33 induces the expression of IL-4, IL-5, and IL-13 and leads to severe pathological changes in mucosal organs. Administration of IL-33 to mice has potent inflammatory effects, including massive blood eosinophilia, increased IL-5 and IgE serum levels, and goblet cell hyperplasia at mucosal surfaces (Schmitz et al. (2005)). Intraperitoneal or intranasal administration of IL-33 to mice led to induction of eosinophilic inflammation in the pulmonary and intestinal mucosa through the IL-13 and STAT6-dependent pathways (Oboki et al. (2010)). Accordingly, IL-33 may play a role in allergic diseases such as asthma, and inflammatory airway diseases, such as chronic obstructive pulmonary disorder (COPD).

It is therefore contemplated that a composition comprising an anti-IL-33 antibody may be useful in the treatment of IL-33-mediated diseases, such as asthma or COPD.

In some instances, the antibody present in the composition of the invention comprises a heavy chain variable domain comprising a VHCDR1 having the sequence of SEQ ID NO: 1, a VHCDR2 having the sequence of SEQ ID NO: 2, a VHCDR3 having the sequence of SEQ ID NO: 3; and a light chain variable domain comprising a VLCDR1 having the sequence of SEQ ID NO: 5, a VLCDR2 having the sequence of SEQ ID NO: 6, and a VLCDR3 having the sequence of SEQ ID NO: 7.

In some instances, the antibody present in the composition of the invention comprises (a) a heavy chain variable domain that is a sequence of amino acids that is at least 95%, 90%, 85% or 80% identical to SEQ ID NO: 4 or a sequence of amino acids encoded by a polynucleotide sequence that is at least 95%, 90%, 85% or 80% identical to SEQ ID NO: 11, (b) a light chain variable domain that is a sequence of amino acids that is at least 80% identical to SEQ ID NO: 8 or a sequence of amino acids encoded by a polynucleotide sequence that is at least 95%, 90%, 85% or 80% identical to SEQ ID NO: 12, or (c) a heavy chain variable domain of (a) and a light chain variable domain of (b).

In some instances, the antibody is a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgG1 antibody, an IgG2 antibody, an IgG3 antibody, and an IgG4 antibody. In some instances, the anti-IL-33 antibody is an IgG1 antibody.

In some instances. the anti-IL-33 antibody comprises a heavy chain variable domain comprising the amino acid sequence of SEQ ID NO: 4. In some instances, the anti-IL-33 antibody comprises a light chain comprising the amino acid sequence of SEQ ID NO: 8. In some instances, the anti-IL-33 antibody comprises a heavy chain variable domain comprising the amino acid sequence of SEQ ID NO: 4 and a light chain comprising the amino acid sequence of SEQ ID NO: 8.

In some instances, the anti-IL-33 antibody comprises a heavy chain comprising the amino acid sequence of SEQ ID NO:9. In some instances, the anti-IL-33 antibody comprises a light chain comprising the amino acid sequence of SEQ ID NO: 10. In some instances, the anti-IL-33 antibody comprises a heavy chain comprising the amino acid sequence of SEQ ID NO:9 and a light chain comprising the amino acid sequence of SEQ ID NO:10.

In some instances, the composition comprises an anti-IL-33 antibody that competes for binding to IL-33 with 33_640087-7B, in an in vitro HTRF competitive binding assay. A antibody is said to competitively inhibit binding of a reference antibody to a given epitope if it specifically binds to that epitope to the extent that it blocks, to some degree, binding of the reference antibody to the epitope. Competitive inhibition may be determined by any method known in the art, for example, solid phase assays such as competition ELISA assays, Dissociation-Enhanced Lanthanide Fluorescent Immunoassays (DELFIA®, Perkin Elmer), and radioligand binding assays. For example, the skilled person could determine whether an antibody thereof competes for binding to IL-33 by using an in vitro competitive binding assay, such as the HTRF assay described in WO2016/156440, paragraphs 881-886, which is incorporated herein by reference. For example, the skilled person could label a first anti-IL-33 antibody with a donor fluorophore and mix multiple concentrations with fixed concentration samples of acceptor fluorophore labelled-redIL-33. Subsequently, the fluorescence resonance energy transfer between the donor and acceptor fluorophore within each sample can be measured to ascertain binding characteristics. To elucidate competitive binding anti-IL-33 antibodies, the skilled person could first mix various concentrations of a test antibody with a fixed concentration of the labelled antibody of Table 6. A reduction in the FRET signal when the mixture is incubated with labelled IL-33 in comparison with a labelled antibody-only positive control would indicate competitive binding to IL-33. A binding molecule or fragment thereof may be said to competitively inhibit binding of the reference antibody to a given epitope by at least 90%, at least 80%, at least 70%, at least 60%, or at least 50%.

In some instances, the composition comprises the anti-IL-33 antibody 33_640087-7B (as described in WO2016/156440, which is herein incorporated by reference). WO2016/156440 discloses that 33_640087-7B binds to redIL-33 with particularly high affinity and attenuates both ST-2 and RAGE-dependent IL-33 signaling.

Other exemplary anti-IL-33 antibodies include ANB020 known as etokimab (as described in WO2015/106080), 9675P (as described in US2014/0271658), A25-3H04 (as described in US2017/0283494), Ab43 (as described in WO2018/081075), IL33-158 (as described in US2018/0037644), 10C12.38.H6. 87Y.581 lgG4 (as described in WO2016/077381) or binding fragments thereof. Other exemplary anti-IL-33 antibodies or antigen binding fragments thereof include any of the other anti-IL-33 antibodies described in WO2016/156440, WO2015/106080, US2014/0271658, US2017/0283494, WO2018/081075, US2018/0037644 or WO2016/077381, all of which are incorporated herein by reference.

Methods of Making

Methods of making the composition of the present disclosure are further provided herein. Accordingly, methods of making a stable, liquid composition having a viscosity of less than about 10 cP and comprising greater than about 100 mg/ml of an anti-IL-33 antibody, at least about 170 mM arginine, such as at least about 190 mM arginine, optionally a surfactant, and a buffer are further provided. In some instances, the method comprises: (i) combining in a solution the antibody, arginine and a buffer, to obtain a solution comprising about 100 mg/mL to about 200 mg/mL anti-IL-33 antibody, at least about 170 mM arginine, such as at least about 170 mM arginine, such as at least about 190 mM arginine, and buffer and (ii) adding a surfactant to the solution to achieve a final surfactant concentration of about 0.03% (w/v)±0.015% (w/v).

Further provided are methods of making a stable, liquid composition having a viscosity of less than about 10 cP and comprising greater than about 100 mg/ml of an anti-IL-33 antibody, at least about 150 mM lysine, optionally a surfactant, and a buffer are further provided. In some instances, the method comprises: (i) combining in a solution the antibody, arginine and a buffer, to obtain a solution comprising about 100 mg/mL to about 200 mg/mL anti-IL-33 antibody, at least about 170 mM arginine, such as at least about 150 mM lysine, and buffer and (ii) adding a surfactant to the solution to achieve a final surfactant concentration of about 0.03% (w/v)±0.015% (w/v).

In some instances, the stable, liquid composition comprises the surfactant at a concentration of 0.03% (w/v)±0.01% (w/v). In some instances, the stable, liquid composition comprises the surfactant at a concentration of about 0.02% (w/v) to about 0.04% (w/v).

In some instances, the stable, liquid composition comprises the surfactant at a concentration of 0.02% (w/v)±0.01% (w/v). In some instances, the stable, liquid composition comprises the surfactant at a concentration of about 0.01% (w/v) to about 0.02% (w/v).

In some instances, the stable, liquid composition comprises about 150 mg/mL of the anti-IL-33 antibody.

In some instances, the stable, liquid composition comprises greater than 170 mM arginine. In some instances, the stable, liquid composition comprises greater than 190 mM arginine. In some instances, the composition comprises less than about 500 mM arginine. For example, the composition comprises about 200 mM, about 210 mM, about 220 mM, about 240 mM, about 260 mM, about 280 mM, about 300 mM, about 350 mM, about 400 mM, or about 450 mM arginine. In some instances, the composition comprises about 220 mM arginine.

In some instances, the stable, liquid composition comprises at least about 150 mM lysine. In some instances, the composition comprises less than about 500 mM lysine. For example, the composition comprises about 160 mM, about 170 mM, about 180 mM, about 200 mM, about 220 mM, about 240 mM, about 260 mM, about 280 mM, about 300 mM, about 350 mM, about 400 mM, or about 450 mM lysine. In some instances, the composition comprises about 170 mM lysine.

In some instances, the viscosity of the stable, liquid composition with the high concentration of arginine is reduced, relative to a liquid composition comprising a lower concentration of the anti-IL-33 antibody, 20 mM histidine, 80 mM arginine, 120 mM sucrose, 0.02% (w/v) polysorbate 80 at pH 6.0 wherein the lower concentration is relative to the concentration of the anti-IL-33 antibody in the stable, liquid composition.

In some instances, the viscosity of the stable, liquid composition is less than about 10 cP. In some instances, the viscosity of the stable, liquid composition is about 9 cP. In some instances, the viscosity is measured at 23° C.

In some instances, reversible self-association (RSA) of the anti-IL-33 antibody within the stable, liquid composition is reduced, relative to the RSA of the anti-IL-33 antibody in a liquid composition comprising a lower concentration of the anti-IL-33 antibody in 20 mM histidine, 80 mM arginine, 120 mM sucrose, 0.02% (w/v) polysorbate 80 at pH 6.0, wherein the lower concentration is relative to the concentration of the anti-IL-33 antibody in the stable, liquid composition.

In some instances, the RSA is reduced 2-fold relative to the RSA of the anti-IL-33 antibody in a liquid composition comprising a lower concentration of the anti-IL-33 antibody in 20 mM histidine, 80 mM arginine, 120 mM sucrose, 0.02% (w/v) polysorbate 80 at pH 6.0.

In some instances, the stable, liquid composition comprises a weight % monomer of at least about 30%, for example, about 30%, about 31%, about 32%, about 33%, about 34%, about 35%, about 36%, about 37%, about 38%, about 39%, about 40%, about 41% about 42%, about 43%, about 44%, about 45%, about 46%, about 47%, about 48%, about 49% or about 50%. In some instances, the stable, liquid composition comprises a weight % monomer of from about 35% to about 50%. In some instances, the stable, liquid composition comprises a weight % monomer of about 40% to about 45%. In some instances, the stable, liquid composition comprises a weight % monomer of from about 35% to about 50% after storage for about 3 months at a temperature from about 2° C. to about 8° C. In some instances, the stable, liquid composition comprises a weight % monomer of from about 35% to about 50% after storage for about 3 months at a temperature from about 2° C. to about 8° C. In some instances, the stable, liquid composition comprises a weight % monomer of from about 40% after storage for about 3 months at a temperature from about 2° C. to about 8° C. In some instances, the stable, liquid composition comprises a weight % monomer of from about 35% to about 50% after storage for about 6 months at a temperature from about 2° C. to about 8° C. In some instances, the stable, liquid composition comprises a weight % monomer of from about 40% after storage for about 6 months at a temperature from about 2° C. to about 8° C.

In some instances, the surfactant is polysorbate 80 or polysorbate 20. In some instances, the surfactant is polysorbate 80.

In some instances, the buffer is made from histidine. In some instances, stable, liquid composition having a final buffer concentration of about 16 mM to about 24 mM, optionally about 17 mM to about 24 mM, optionally about 18 mM to about 24 mM.

In some instances, stable, liquid composition has a pH of about pH 5.5.

In some instances, the anti-IL-33 antibody is any of those described herein. In some instances, the anti-IL-33 antibody is 33_640087-7B.

Articles of Manufacture, Syringes, and Vials

The present disclosure provides an article of manufacture comprising any one of the presently disclosed compositions, optionally, comprising about 0.5 mL to about 5 mL (e.g., about 0.5 mL to about 4.5 mL, about 0.5 mL to about 4 mL, about 0.5 mL to about 3.5 mL, about 0.5 mL to about 3 mL, about 0.5 mL to about 2.5 mL, about 0.5 mL to about 2 mL, about 0.5 mL to about 1.5 mL, about 0.5 mL to about 1 mL, about 1 mL to about 5 mL, about 1.5 mL to about 5 mL, about 2 mL to about 5 mL, about 2.5 mL to about 5 mL, about 3 mL to about 5 mL, about 3.5 mL to about 5 mL, about 4 mL to about 5 mL, about 4.5 mL to about 5 mL) of the composition. In some instances, the composition comprises greater than about 100 mg/mL of an anti-IL33 antibody (e.g., 33_640087-7B). In some instances, the composition comprises about 130 mg/mL to about 170 mg/mL anti-IL-33 antibody (e.g., 33_640087-7B), about 0.03% (w/v)±0.015% (w/v) polysorbate 80, about 220 mM arginine, and about 16 mM to about 24 mM histidine, wherein the pH is less than about 6, optionally, about pH 5.5. Optionally, the pH is from about Ph 5.0 to about pH 6.0.

The present disclosure also provides a pre-filled syringe (PFS) comprising any one of the presently disclosed compositions, optionally, comprising about 0.5 mL to about 5 mL (e.g., about 0.5 mL to about 4.5 mL, about 0.5 mL to about 4 mL, about 0.5 mL to about 3.5 mL, about 0.5 mL to about 3 mL, about 0.5 mL to about 2.5 mL, about 0.5 mL to about 2 mL, about 0.5 mL to about 1.5 mL, about 0.5 mL to about 1 mL, about 1 mL to about 5 mL, about 1.5 mL to about 5 mL, about 2 mL to about 5 mL, about 2.5 mL to about 5 mL, about 3 mL to about 5 mL, about 3.5 mL to about 5 mL, about 4 mL to about 5 mL, about 4.5 mL to about 5 mL) of the composition. In some instances, the composition comprises the composition comprises greater than about 100 mg/mL of an anti-IL33 antibody (e.g., 33_640087-7B). In some instances, the composition comprises about 130 mg/mL to about 170 mg/mL anti-IL-33 antibody (e.g., 33_640087-7B), about 0.03% (w/v)±0.015% (w/v) polysorbate 80, about 220 mM arginine, and about 16 mM to about 24 mM histidine, wherein the pH is less than about 6, optionally, about pH 5.5.

Also provided is a vial comprising any one of the presently disclosed compositions, optionally, comprising about 0.5 mL to about 5 mL (e.g., about 0.5 mL to about 4.5 mL, about 0.5 mL to about 4 mL, about 0.5 mL to about 3.5 mL, about 0.5 mL to about 3 mL, about 0.5 mL to about 2.5 mL, about 0.5 mL to about 2 mL, about 0.5 mL to about 1.5 mL, about 0.5 mL to about 1 mL, about 1 mL to about 5 mL, about 1.5 mL to about 5 mL, about 2 mL to about 5 mL, about 2.5 mL to about 5 mL, about 3 mL to about 5 mL, about 3.5 mL to about 5 mL, about 4 mL to about 5 mL, about 4.5 mL to about 5 mL) of the composition. In some instances, the composition comprises the composition comprises greater than about 100 mg/mL of an anti-IL33 antibody (e.g., 33_640087-7B). In some instances, the composition comprises about 130 mg/mL to about 170 mg/mL anti-IL-33 antibody (e.g., 33_640087-7B), about 0.03% (w/v)±0.015% (w/v) polysorbate 80, about 220 mM arginine, and about 16 mM to about 24 mM histidine, wherein the pH is less than about 6, optionally, about pH 5.5.

Kits

The present disclosure also provides a kit including a composition described herein together with a package insert, package label, instructions, or other labeling directing or disclosing any of the methods disclosed herein. In certain instances, the present disclosure provides kits for producing a single-dose administration unit. In certain instances of this disclosure, kits containing single and multi-chambered pre-filled syringes (e.g., liquid syringes) are included.

Methods of Treatment

The disclosure also provides the use of 33_640087-7B, or another anti-IL-33 antibody disclosed herein, or an antigen binding portion thereof, in the manufacture of a medicament for treating a subject with an IL-33-mediated disease.

Provided herein are methods for treating an IL-33-mediated disease in a subject. In some instances, the disclosure provides the compositions disclosed herein for use in the treatment of an IL-33-mediated disease in a subject. Provided herein, the disclosure also provides for the use of an anti-IL-33 antibody in the manufacture of a medicament for treating a subject with an IL-33-mediated disease, wherein the medicament comprises any composition disclosed herein.

In some instances, the methods, compositions for use, or uses provide herein, are for the treatment of an IL-33-mediated disorder selected from asthma, atopic dermatitis and chronic obstructive pulmonary disorder.

In some instances, the methods, compositions for use, or uses provide herein, are for the treatment of diabetic kidney disease.

In some instances, the subject is human.

### Embodiments

1. A composition comprising greater than about 100 mg/ml of an anti-IL-33 antibody, at least 170 mM arginine or at least 150 mM lysine and a buffer, wherein the anti-IL-33 antibody comprises:
   i. a heavy chain variable domain comprising a VHCDR1 having the sequence of SEQ ID NO: 1, a VHCDR2 having the sequence of SEQ ID NO: 2, a VHCDR3 having the sequence of SEQ ID NO: 3; and
   ii. a light chain variable domain comprising a VLCDR1 having the sequence of SEQ ID NO: 5, a VLCDR2 having the sequence of SEQ ID NO: 6, and a VLCDR3 having the sequence of SEQ ID NO: 7.
2. The composition of embodiment 1, wherein the anti-IL-33 antibody comprises:
   i. a heavy chain variable domain that is:
      i. a sequence of amino acids that is at least 95%, 90%, 85% or 80% identical to SEQ ID NO: 4; or
      ii. a sequence of amino acids encoded by a polynucleotide sequence that is at least 80% identical to SEQ ID NO: 11;
   ii. a light chain variable domain that is:
      i. a sequence of amino acids that is at least 95%, 90%, 85% or 80% identical to SEQ ID NO: 8; or
      ii. a sequence of amino acids encoded by a polynucleotide sequence that is at least 80% identical to SEQ ID NO: 12; or
   iii. a heavy chain variable domain of (a) and a light chain variable domain of (b).
3. The composition of embodiment 1 or 2, wherein the anti-IL-33 antibody is an IgG1 antibody.
4. The composition of any preceding embodiment, wherein the anti-IL-33 antibody comprises a heavy chain variable domain comprising the amino acid sequence of SEQ ID NO: 4, a light chain comprising the amino acid sequence of SEQ ID NO: 8, or a heavy chain variable domain comprising the amino acid sequence of SEQ ID NO: 4 and a light chain comprising the amino acid sequence of SEQ ID NO: 8.
5. The composition of any preceding embodiment, wherein the anti-IL-33 antibody comprises a heavy chain comprising the amino acid sequence of SEQ ID NO:9, a light chain comprising the amino acid sequence of SEQ ID NO:10, or a heavy chain comprising the amino acid sequence of SEQ ID NO:9 and a light chain comprising the amino acid sequence of SEQ ID NO:10.
6. The composition of any preceding embodiment, wherein the anti-IL-33 antibody competes for binding to IL-33 with 33_640087-7B, in an in vitro HTRF competitive binding assay.
7. The composition of any preceding embodiment, wherein the anti-IL-33 antibody is present at a concentration less than about 200 mg/ml.
8. The composition of any preceding embodiment, wherein the anti-IL-33 antibody is present at a concentration less than about 180 mg/ml.
9. The composition of any preceding embodiment, wherein the anti-IL-33 antibody is present at a concentration less than about 160 mg/ml.
10. The composition of any preceding embodiment, wherein the anti-IL-33 antibody is present at a concentration of about 100 mg/ml to about 200 mg/ml.
11. The composition of any preceding embodiment, wherein the anti-IL-33 antibody is present at a concentration of about 130 mg/ml to about 170 mg/ml.
12. The composition of any preceding embodiment, wherein the anti-IL-33 antibody is present at a concentration of 150 mg/ml±10%.
13. The composition of any preceding embodiment, further comprising a surfactant.
14. The composition of any embodiment 13, wherein the surfactant is amphipathic and anionic.
15. The composition of embodiment 14, wherein the surfactant is polysorbate.
16. The composition of embodiment 15, wherein the surfactant is polysorbate 20 or polysorbate 80 or a mixture thereof
17. The composition of any one of embodiments 13 to 16, wherein the surfactant is at a concentration of about 0.005% (w/v) to about 0.05% (w/v).
18. The composition of embodiment 17, comprising 0.03% (w/v)±0.010% (w/v) surfactant.
19. The composition of embodiment 17, comprising about comprising about 0.015% (w/v), 0.03% (w/v), or 0.045% (w/v) surfactant.
20. The composition of any one of embodiments 16 to 19, wherein the surfactant is polysorbate 80.
21. The composition of any preceding embodiment, comprising at least about 190 mM arginine.
22. The composition of any preceding embodiment, comprising about 190 mM to about 250 mM arginine.
23. The composition of any preceding embodiment, comprising about 220 mM arginine.
24. The composition of any preceding embodiment, wherein the arginine is L-arginine hydrochloride.
25. The composition according to any of embodiments 1 to 20, comprising about 150 mM to about 250 mM lysine.
26. The composition according to embodiment 25, comprising about 170 mM lysine.
27. The composition according to any of embodiments 1-20, 25 or 26, wherein the lysine is L-lysine.
28. The composition of any preceding embodiment, wherein the buffer is succinate, histidine or acetate.
29. The composition of embodiment 28, wherein the buffer is histidine.
30. The composition of embodiment 29, wherein the buffer is L-histidine/L-histidine hydrochloride.
31. The composition of any preceding embodiment, wherein the buffer is at a concentration of about 10 mM to about 30 mM.
32. The composition of embodiment 31, wherein the buffer is at a concentration of about 16 mM to about 24 mM, optionally about 17 mM to about 24 mM, optionally about 18 mM to about 24 mM.
33. The composition of either of embodiments 31 or 32, wherein the buffer is at a concentration of about 19 mM to about 21 mM.
34. The composition of any preceding embodiment, comprising 20 mM±10% buffer.
35. The composition of any preceding embodiment, which is a liquid.
36. The composition of any preceding embodiment, wherein the pH is less than about pH 6.0.
37. The composition of any preceding embodiment, wherein the pH is about pH 5.0 to about pH 6.0.
38. The composition of any preceding embodiment, wherein the pH is about pH 5.2, about pH 5.5, or about pH 5.8.
39. The composition of any preceding embodiment, wherein the pH is about pH 5.5.
40. The composition of any one of embodiments 35 to 39, characterized by a reduced viscosity, relative to a composition comprising less than or equal to 80 mM arginine.
41. The composition of any one of embodiments 35 to 39, characterized by a reduced viscosity, relative to a composition comprising a lower concentration of the anti-IL-33 antibody, 20 mM histidine, 80 mM arginine, 120 mM sucrose, 0.02% (w/v) polysorbate 80 at pH 6.0.
42. The composition of any one of embodiments 35 to 41, characterized by a viscosity of less than about 10 cP at 23° C. wherein the concentration of the anti-IL-33 antibody is about 150 mg/ml 10%.
43. The composition of any of embodiments 35 to 41, wherein the viscosity is from about 5 cP to about 20 cP, optionally less than about 10 cP, such as about 9 cP.
44. The composition of any one of embodiments 35 to 43, characterized by the anti-IL-33 antibody having reduced reversible self-association relative to the reversible self-association of the anti-IL-33 antibody in a composition comprising 20 mM histidine, 80 mM arginine, 120 mM sucrose, 0.02% (w/v) polysorbate 80 at pH 6.0 and a lower concentration of the anti-IL-33 antibody, for example 50 mg/ml.
45. The composition of any of embodiments 35 to 44, comprising a weight % monomer of about 35% to about 50%.
46. The composition of any preceding embodiment, wherein less than about 5%, optionally less than about 2%, of the antibody aggregates after about 12 months to about 18 months of storage between about 2° C. to about 8° C., as measured by size exclusion chromatography (SEC).
47. A composition comprising about 130 mg/ml to about 170 mg/ml 33_640087-7B, about 0.03% (w/v)±0.015% polysorbate 80, about 220 mM arginine, and about 16 to about 24 mM histidine buffer, wherein the pH is pH 5.5±0.5.
48. The composition of embodiment 47, comprising about 150 mg/ml 33_640087-7B.
49. The composition of either of embodiments 47 or 48, comprising about 18 mM to about 22 mM histidine buffer, optionally about 20 mM histidine buffer.
50. The composition of any one of embodiments 47 to 49, wherein the arginine is L-arginine hydrochloride.
51. A composition comprising about 130 mg/ml to about 170 mg/ml of an anti-IL-33 antibody, about 0.03% (w/v)±0.015% polysorbate 80, about 220 mM arginine, and about 16 to about 24 mM histidine buffer, wherein the pH is pH 5.5±0.5.
52. The composition of any of embodiments 47 to 51, which is a liquid.
53. An article of manufacture, comprising the composition of any preceding embodiment, optionally comprising 0.5 ml to about 5 ml (e.g., 1 ml to about 3 ml) of the composition.
54. A vial comprising the composition of any of embodiments 1 to 52, optionally comprising about 0.5 ml to about 5 ml (e.g., 1 ml to about 3 ml) of the composition.
55. A method of treating an IL-33-mediated disorder in a subject comprising administering to the subject a therapeutically effective amount of the composition of any one of embodiments 1 to 52.
56. The method of embodiment 55, wherein the IL-33-mediated disorder is asthma, atopic dermatitis or chronic obstructive pulmonary disorder.
57. The method of embodiment 55, wherein the IL-33-mediated disorder is diabetic kidney disease.
58. A method of making a stable, liquid composition having a viscosity of less than about 10 cP, and comprising greater than about 100 mg/ml of an anti-IL-33 antibody, at least about 170 mM arginine or about 150 mM lysine, a surfactant and a buffer, comprising the steps of:
   i. combining in a solution the antibody, arginine and a buffer, to obtain a solution comprising about 100 mg/mL to about 200 mg/mL anti-IL-33 antibody, at least about 170 mM arginine or about 150 mM lysine, and buffer; and
   ii. adding a surfactant to the solution to achieve a final surfactant concentration of about 0.03% (w/v)±0.015% (w/v).
59. The method of embodiment 58, wherein the stable, liquid composition comprises greater than 190 mM arginine.
60. The method of either of embodiments 58 or 59, wherein the stable, liquid composition comprises about 220 mM arginine.
61. The composition of any one of embodiments 58 to 60, wherein the arginine is L-arginine hydrochloride.
62. The method of any one of embodiments 58 to 61, wherein reversible self-association (RSA) of the anti-IL-33 antibody within the stable, liquid composition is reduced, relative to the RSA of the anti-IL-33 antibody in a liquid composition comprising a lower concentration of the anti-IL-33 antibody in 20 mM histidine, 80 mM arginine, 120 mM sucrose, 0.02% (w/v) polysorbate 80 at pH 6.0.
63. The method of any of embodiments 58 to 62, wherein, the viscosity of the liquid formulation is reduced, relative to a liquid formulation comprising a lower concentration of the anti-IL-33 antibody, 20 mM histidine, 80 mM arginine, 120 mM sucrose, 0.02% (w/v) polysorbate 80 at pH 6.0.
64. The method of any of embodiments 58 to 63, wherein the surfactant is polysorbate 80.
65. The method of any of embodiments 58 to 64, wherein the buffer is made from histidine.
66. The method of embodiments 65, wherein the method results in a stable, liquid formulation having a final buffer concentration of about 16 mM to about 24 mM, optionally about 17 mM to about 24 mM, optionally about 18 mM to about 24 mM.
67. The method of any of embodiments 58 to 66, the method results in a stable, liquid formulation having a pH of from about pH 5 to about pH 6, optionally about pH 5.5.
68. The method of any one of embodiments 58 to 67, wherein the anti-IL-33 antibody is as defined in any of embodiments 1 to 5.
69. The method of any one of embodiments 58 to 68, wherein cP is measured at 23° C.

### Examples

Generation of a Formulation with Improved Characteristics

33_640087-7B is a human immunoglobulin (Ig) G1 monoclonal antibody (mAb) that binds to human interleukin (IL)-33, prevents binding of IL-33 to its receptor ST2, and inhibits conversion to disulfide-bonded (DSB) IL 33.

A Phase 1 clinical study of 33_640087-7B (Study D9180C00001) has been completed. 33_640087-7B was found to be generally safe and well tolerated, and there were no safety concerns following administration up to 300 mg 33_640087-7B intravenous (IV). 33_640087-7B was supplied vialed as a lyophilized powder. Each vial comprised a nominal 50 mg of 33_640087-7B. Upon reconstitution with 1.2 mL of sterile water for injection, the solution contained 50 mg/mL 33_640087-7B in 20 mM L histidine/L histidine-hydrochloride, 80 mM L-arginine-hydrochloride, 120 mM sucrose, 0.02% (weight/volume [w/v]) polysorbate 80, pH 6.0 (the “Phase I formulation”).

This example describes the surprising outcome of the effort to reformulate 33_640087-7B to achieve a higher unit dose per ml of composition for subsequent clinical studies.

The reversible self-association characteristics of 33_640087-7B in the Phase I formulation was measured by obtaining the weight % monomer and soluble, reversible higher order species using static light scattering. Static light scattering intensity in volts is measured as a function of concentration (mg/mL) and which is converted into apparent molecular weight (kDa) using the Rayleigh equation. Weight fractions of the monomer and soluble, reversible higher order species were extracted from the measured apparent molecular weight using effective hard particle model (Fernandez and Minton (2009) Biophys J 96:1992-8).

Table 1 shows the reversible self-association characteristics of the aqueous Phase I composition:

| | 20 mM His/His-HCl, |
|---|---|
| | 80 mM Arg-HCl, |
| | 120 mM sucrose, |
| | 0.02% (w/v) PS80, |
| Composition | pH 6.0 |
| 33_640087-7B | 50 mg/ml |
| concentration | |
| % soluble, | 22.4% Monomer, 77.6% |
| higher order | RSA, (19.2% dimer, |
| species. | 32.8% tetramer, 14.3% |
| | octamer and 11.3% dodecamer) |

Next, the concentration of 33_640087-7B was increased 3-fold to 150 mg/ml, and the viscosity was measured at 23° C. The viscosity was determined to be about 24 centiPoise.

Attempts were next made to reformulate 33_640087-7B. It was unexpectedly found that altering the excipient amounts in the composition led to a formulation with improved RSA characteristics in comparison to the Phase 1 formulation.

Table 2 shows the reversible self-association characteristics of the aqueous Phase I and a next generation composition.

| | 20 mM His/His-HCl, | |
|---|---|---|
| | 80 mM Arg-HCl, | 20 mM His/His-HCl, |
| | 120 mM sucrose, | 220 mM Arg-HCl, |
| | 0.02% (w/v) PS80, | 0.03% (w/v) PS80, |
| Composition | pH 6.0 | pH 5.5 |
| 33_640087-7B | 50 mg/ml | 150 mg/ml |
| concentration | | |
| % soluble, | 22.4% Monomer, 77.6% | 44% Monomer, 56% |
| higher order | RSA, (19.2% dimer, | RSA, (36% Trimer, |
| species. | 32.8% tetramer, 14.3% | 20% hexamer) |
| | octamer and 11.3% dodecamer) | |

The reduction in RSA led to a significant improvement in viscosity measured at 23° C. (about 9 cP), relative to the viscosity of the Phase 1 composition comprising an equivalent antibody concentration (about 24° cP). Notably, this improvement was observed at three times the protein concentration.

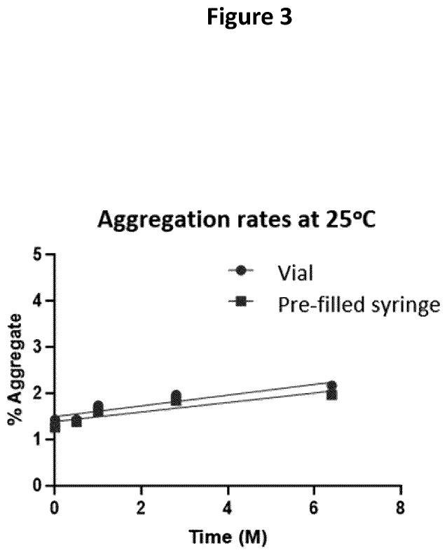

It was also found that the RSA characteristics were stable long-term. The % distribution of soluble, higher order species did not change significantly when stored between 2 and 8° C. over a period of 6 months. RSA stability was observed in the next generation composition at multiple antibody concentrations (FIG. 3).

Table 3 shows the weight 00 distribution of monomer, and trimer and hexamer (i.e., soluble, higher order species) over time.

| | Weight | Weight | Weight |
|---|---|---|---|
| Sample Description | % monomer | % trimer | % hexamer |
| T = 0 | 44 | 36 | 20 |
| 3 months at 2-8° C. | 40 | 43 | 17 |
| 6 months at 2-8° C. | 41 | 40 | 19 |

The long-term stability of the next generation composition was tested at multiple temperatures at multiple time points. Stability data was generated for compositions stored in either glass vials or pre-filled syringes.

Table 4 shows the long-term stability characteristics as a function of insoluble aggregate formation expressed as a percentage of the original antibody concentration (about 150 mg/mi).

| Time | Vial | PFS | | | | |
|---|---|---|---|---|---|---|
| (months - | (2 to | (2 to | Vial | PFS | Vial | PFS |
| M) | 8° C.) | 8° C.) | (25° C.) | (25° C.) | (40° C.) | (40° C.) |
| 0 | 1.3 | 1.3 | 1.4 | 1.3 | 1.8 | 1.3 |
| 0.5 | 1.3 | 1.3 | 1.4 | 1.4 | 1.9 | 1.8 |
| 1 | 1.3 | 1.3 | 1.7 | 1.6 | 2.6 | 2.4 |
| 2.8 | 1.5 | 1.5 | 2.0 | 1.8 | 4.1 | |
| 6.4 | 1.5 | 1.5 | 2.2 | 2.0 | | |
| 12.4 | 1.3 | 1.3 | | | | |
| 18.1 | 1.5 | 1.5 | | | | |

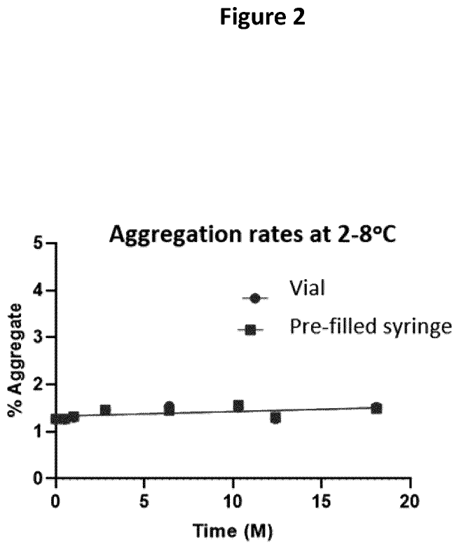

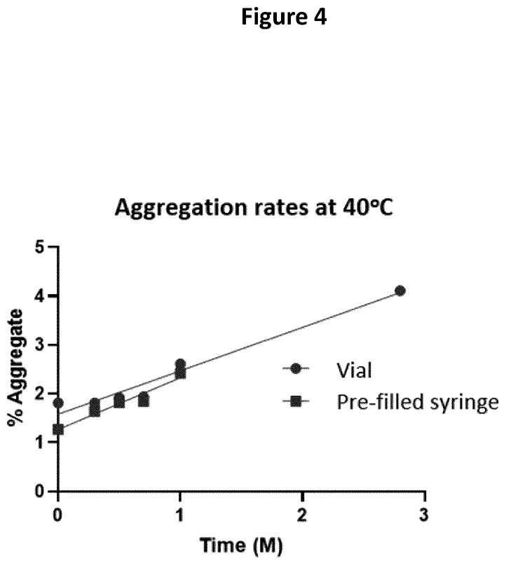

This data is illustrated graphically in FIGS. 2, 3 and 4.

Next, a univariate analysis of viscosity (Cp) was carried out across multiple 33-640087_7B and arginine concentrations (the remaining formulation components were fixed).

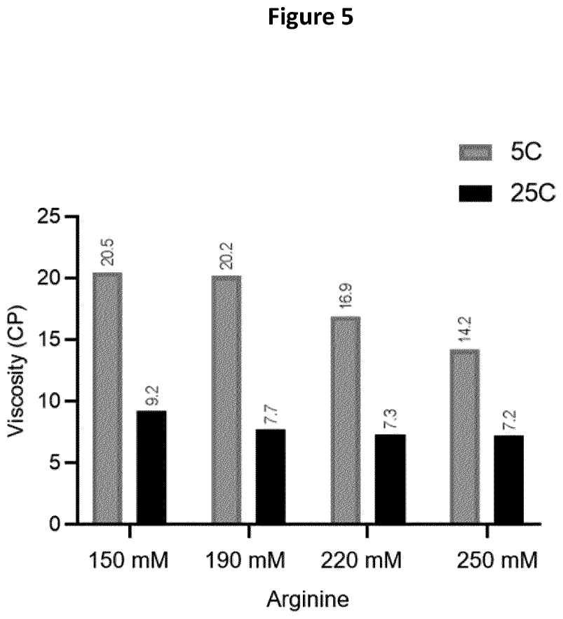

The results are shown in FIG. 5. At multiple high arginine concentrations (150 mM, 190 mM, 220 mM and 250 mM), the Cp at 25° C. was significantly lower that the Cp calculated for the Phase 1 formulation at 23° C. (24 Cp). The Cp at 5° C. improved with increasing concentrations of arginine.

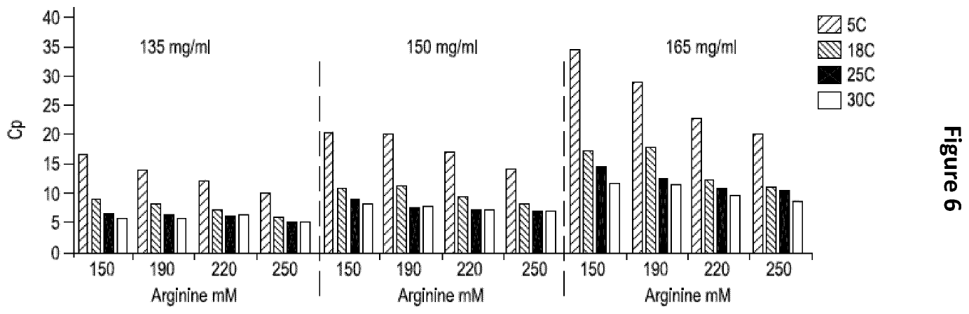

Cp was also test at multiple protein concentrations (135 mg/ml, 150 mg/ml and 165 mg/ml). The results are shown in FIG. 6. Cp was measured at multiple temperatures (5° C., 18° C., 25° C. and 30° C.). The analysis reveals that even at high protein concentrations, Cp decreases at increasing concentrations of arginine, particularly at temperatures of greater than 18° C. Generally when at least 190 mM arginine is used the viscosity at about 25° C. is at or under the desired 10 Cp value when the formulation comprises 165 mg/ml 33-640087_7B. 33-640087_7B formulation robustness was also analyzed at several temperatures, pHs, arginine concentrations and excipient concentrations. Formulations were stored at either 40° C., 25° C. or 2-8° C., for 1, 6 or 11 months respectively. Samples were taken at multiple timepoints under each condition and aggregate formation was determined using standard analytical techniques. The percent aggregate formation per month was calculated. Slope gradients from the resulting plots are shown in Table 5. The results show that aggregation rate is stable within the range of pH, surfactant or arginine concentration under the conditions tested. A slight increase in the rate of aggregation was observed at pH 5.0 in one month accelerated stability conditions (40° C.).

TABLE 5

| | | pH 5.0 220 | pH 6.0 220 | pH 5.5 190 | pH 5.5 270 | pH 5.5 | pH 5.5 | pH 5.5 |
|---|---|---|---|---|---|---|---|---|
| | | mM Arg- | mM Arg- | mM Arg- | mM Arg- | 0.02% | 0.03% | 0.05% |
| | | HCL | HCL | HCL | HCL | PS80 | PS80 | PS80 |
| 40° C. | Slope (%/m) | 1.7 | 0.9 | 0.9 | 1.0 | 1.1 | 1.1 | 1.4 |
| 25° C. | Slope (%/m) | 0.2 | 0.2 | 0.2 | 0.1 | 0.1 | 0.1 | 0.2 |
| 2-8° C. | Slope (%/m) | 0.02 | 0.03 | 0.03 | 0.02 | 0.03 | 0.04 | 0.05 |

Multiple additional formulations were tested. Of particular interest, a formulation comprising 20 mM Histidine/Histidine-HCl, 170 mM Lysine-HCl, 0.02% PS80, pH 5.5 and about 160 mg/ml 33-640087_7B also showed significant improvements in Cp at 23° C. (8.7 Cp). Analysis of Cp at 23° C. of formulations comprising 150 mg/ml 33-640087_7B and either 150 mM or 190 mM lysine (and 20 mM Histidine/Histidine-HCl, 0.02% PS80, pH 5.5) also displayed significant improvements in viscosity, i.e., a Cp of less than 10, compared to the Cp of the phase 1 formulation.

### CONCLUSION

A next generation formulation has been developed that leads to a surprising reduction in RSA at high antibody concentrations. The next generation formulation may allow administration of a greater unit dose per volume of an anti-IL-33 antibody, enabling a greater therapeutic dose to be administered. This may reduce patient discomfort, for example, for subcutaneous delivery, by reducing the volume of drug product to be delivered at the injection site, leading to increased patient compliance. It may also enable a greater dynamic range of doses to be explored in the clinic, increasing the prospect of discovering the most therapeutically effective dose.

In addition, the formulation has an acceptable long-term stability profile and reduced viscosity. Reducing viscosity may positively impact on drug administration by increasing the functionality of the device used to administer the antibody. Similarly, the ability of a health care provider or patient to manually inject the drug into the patient may be improved by low viscosity.

SEQUENCES

SEQ ID NO 1: 33_640087-7B VH CDR1
Ser Tyr Ala Met Ser
SEQ ID NO 2: 33_640087-7B VH CDR2
Gly Ile Ser Ala Ile Asp Gln Ser Thr Tyr Tyr Ala Asp Ser Val Lys Gly
SEQ ID NO 3: 33_640087-7B VH CDR3
Gln Lys Phe Met Gln Leu Trp Gly Gly Gly Leu Arg Tyr Pro Phe Gly Tyr
SEQ ID NO 4: 33_640087-7B VH
Glu Val Gln Leu Leu Glu Ser Gly Gly Gly Leu Val Gln Pro Gly Gly
Ser Leu Arg Leu Ser Cys Ala Ala Ser Gly Phe Thr Phe Ser Ser Tyr
Ala Met Ser Trp Val Arg Gln Ala Pro Gly Lys Gly Leu Glu Trp Val
Ser Gly Ile Ser Ala Ile Asp Gln Ser Thr Tyr Tyr Ala Asp Ser Val
Lys Gly Arg Phe Thr Ile Ser Arg Asp Asn Ser Lys Asn Thr Leu Tyr
Leu Gln Met Asn Ser Leu Arg Ala Glu Asp Thr Ala Val Tyr Tyr Cys
Ala Arg Gln Lys Phe Met Gln Leu Trp Gly Gly Gly Leu Arg Tyr Pro
Phe Gly Tyr Trp Gly Gln Gly Thr Met Val Thr Val Ser Ser
SEQ ID NO: 5 33_640087-7B VL CDR1
Ser Gly Glu Gly Met Gly Asp Lys Tyr Ala Ala
SEQ ID NO 6: 33_640087-7B VL CDR2
Arg Asp Thr Lys Arg Pro Ser
SEQ ID NO 7: 33_640087-7B VL CDR3
Gly Val Ile Gln Asp Asn Thr Gly Val
SEQ ID NO 8: 33_640087-7B VL
Ser Tyr Val Leu Thr Gln Pro Pro Ser Val Ser Val Ser Pro Gly Gln
Thr Ala Ser Ile Thr Cys Ser Gly Glu Gly Met Gly Asp Lys Tyr Ala
Ala Trp Tyr Gln Gln Lys Pro Gly Gln Ser Pro Val Leu Val Ile Tyr
Arg Asp Thr Lys Arg Pro Ser Gly Ile Pro Glu Arg Phe Ser Gly Ser
Asn Ser Gly Asn Thr Ala Thr Leu Thr Ile Ser Gly Thr Gln Ala Met
Asp Glu Ala Asp Tyr Tyr Cys Gly Val Ile Gln Asp Asn Thr Gly Val
Phe Gly Gly Gly Thr Lys Leu Thr Val Leu
SEQ ID NO 9: 33_640087-7B HC
Glu Val Gln Leu Leu Glu Ser Gly Gly Gly Leu Val Gln Pro Gly Gly
Ser Leu Arg Leu Ser Cys Ala Ala Ser Gly Phe Thr Phe Ser Ser Tyr
Ala Met Ser Trp Val Arg Gln Ala Pro Gly Lys Gly Leu Glu Trp Val
Ser Gly Ile Ser Ala Ile Asp Gln Ser Thr Tyr Tyr Ala Asp Ser Val
Lys Gly Arg Phe Thr Ile Ser Arg Asp Asn Ser Lys Asn Thr Leu Tyr
Leu Gln Met Asn Ser Leu Arg Ala Glu Asp Thr Ala Val Tyr Tyr Cys
Ala Arg Gln Lys Phe Met Gln Leu Trp Gly Gly Gly Leu Arg Tyr Pro
Phe Gly Tyr Trp Gly Gln Gly Thr Met Val Thr Val Ser Ser Ala Ser
Thr Lys Gly Pro Ser Val Phe Pro Leu Ala Pro Ser Ser Lys Ser Thr
Ser Gly Gly Thr Ala Ala Leu Gly Cys Leu Val Lys Asp Tyr Phe Pro
Glu Pro Val Thr Val Ser Trp Asn Ser Gly Ala Leu Thr Ser Gly Val
His Thr Phe Pro Ala Val Leu Gln Ser Ser Gly Leu Tyr Ser Leu Ser
Ser Val Val Thr Val Pro Ser Ser Ser Leu Gly Thr Gln Thr Tyr Ile
Cys Asn Val Asn His Lys Pro Ser Asn Thr Lys Val Asp Lys Arg Val
Glu Pro Lys Ser Cys Asp Lys Thr His Thr Cys Pro Pro Cys Pro Ala
Pro Glu Leu Leu Gly Gly Pro Ser Val Phe Leu Phe Pro Pro Lys Pro
Lys Asp Thr Leu Met Ile Ser Arg Thr Pro Glu Val Thr Cys Val Val
Val Asp Val Ser His Glu Asp Pro Glu Val Lys Phe Asn Trp Tyr Val
Asp Gly Val Glu Val His Asn Ala Lys Thr Lys Pro Arg Glu Glu Gln
Tyr Asn Ser Thr Tyr Arg Val Val Ser Val Leu Thr Val Leu His Gln
Asp Trp Leu Asn Gly Lys Glu Tyr Lys Cys Lys Val Ser Asn Lys Ala
Leu Pro Ala Pro Ile Glu Lys Thr Ile Ser Lys Ala Lys Gly Gln Pro
Arg Glu Pro Gln Val Tyr Thr Leu Pro Pro Ser Arg Glu Glu Met Thr
Lys Asn Gln Val Ser Leu Thr Cys Leu Val Lys Gly Phe Tyr Pro Ser
Asp Ile Ala Val Glu Trp Glu Ser Asn Gly Gln Pro Glu Asn Asn Tyr
Lys Thr Thr Pro Pro Val Leu Asp Ser Asp Gly Ser Phe Phe Leu Tyr
Ser Lys Leu Thr Val Asp Lys Ser Arg Trp Gln Gln Gly Asn Val Phe
Ser Cys Ser Val Met His Glu Ala Leu His Asn His Tyr Thr Gln Lys
Ser Leu Ser Leu Ser Pro Gly Lys
SEQ ID NO 10: 33_640087-7B LC
Ser Tyr Val Leu Thr Gln Pro Pro Ser Val Ser Val Ser Pro Gly Gln
Thr Ala Ser Ile Thr Cys Ser Gly Glu Gly Met Gly Asp Lys Tyr Ala
Ala Trp Tyr Gln Gln Lys Pro Gly Gln Ser Pro Val Leu Val Ile Tyr
Arg Asp Thr Lys Arg Pro Ser Gly Ile Pro Glu Arg Phe Ser Gly Ser
Asn Ser Gly Asn Thr Ala Thr Leu Thr Ile Ser Gly Thr Gln Ala Met
Asp Glu Ala Asp Tyr Tyr Cys Gly Val Ile Gln Asp Asn Thr Gly Val
Phe Gly Gly Gly Thr Lys Leu Thr Val Leu Gly Gln Pro Lys Ala Ala
Pro Ser Val Thr Leu Phe Pro Pro Ser Ser Glu Glu Leu Gln Ala Asn
Lys Ala Thr Leu Val Cys Leu Ile Ser Asp Phe Tyr Pro Gly Ala Val
Thr Val Ala Trp Lys Ala Asp Ser Ser Pro Val Lys Ala Gly Val Glu
Thr Thr Thr Pro Ser Lys Gln Ser Asn Asn Lys Tyr Ala Ala Ser Ser
Tyr Leu Ser Leu Thr Pro Glu Gln Trp Lys Ser His Arg Ser Tyr Ser
Cys Gln Val Thr His Glu Gly Ser Thr Val Glu Lys Thr Val Ala Pro
Thr Glu Cys Ser
SEQ ID NO 11: 33_640087-7B VH DNA
gaggtgcagc tgttggagtc tgggggaggc ttggtacagc ctggggggtc cctgagactc
tcctgtgcag cctctggatt cacctttagc agctatgcca tgagctgggt ccgccaggct
ccagggaagg ggctggagtg ggtctcaggc atttctgcaa tagaccaaag cacatactac
gcagactccg tgaagggccg gttcaccatc tccagagaca attccaagaa cacgctgtat
ctgcaaatga acagcctgag agccgaggac acggccgtgt attactgtgc ccggcagaag
ttcatgcagc tatggggggg gggcttgcgt tatcccttcg gctactgggg ccaggggaca
atggtcaccg tctcctca
SEQ ID NO 12: 33_640087-7B VL DNA
tcctatgtgc tgactcagcc accctcagtg tccgtgtccc caggacagac ggccagcatc
acctgctctg gagaaggaat gggggataaa tatgctgcct ggtatcagca gaagccaggc
cagtcacctg tgctggtcat ctatcgagat acaaagcggc cctcagggat ccctgagcga
ttctctggct ccaactctgg gaacacagcc acgttgacca tcagcgggac ccaggctatg
gatgaggctg actattactg tggggtgatc caggacaaca ctggggtatt cggcggaggg
accaagctca ccgtccta

1. A composition comprising greater than about 100 mg/ml of an anti-IL-33 antibody, at least 170 mM arginine or at least 150 mM lysine and a buffer, wherein the anti-IL-33 antibody comprises:
i. a heavy chain variable domain comprising a VHCDR1 having the sequence of SEQ ID NO: 1, a VHCDR2 having the sequence of SEQ ID NO: 2, a VHCDR3 having the sequence of SEQ ID NO: 3; and
ii. a light chain variable domain comprising a VLCDR1 having the sequence of SEQ ID NO: 5, a VLCDR2 having the sequence of SEQ ID NO: 6, and a VLCDR3 having the sequence of SEQ ID NO: 7.

2. The composition of claim 1, wherein the anti-IL-33 antibody comprises:
i. a heavy chain variable domain that is:
i. a sequence of amino acids that is at least 95%, 90%, 85% or 80% identical to SEQ ID NO: 4; or
ii. a sequence of amino acids encoded by a polynucleotide sequence that is at least 80% identical to SEQ ID NO: 11;
ii. a light chain variable domain that is:
i. a sequence of amino acids that is at least 95%, 90%, 85% or 80% identical to SEQ ID NO: 8; or
ii. a sequence of amino acids encoded by a polynucleotide sequence that is at least 80% identical to SEQ ID NO: 12; or
iii. a heavy chain variable domain of (a) and a light chain variable domain of (b).

3. The composition of claim 1 or 2, wherein the anti-IL-33 antibody is an IgG1 antibody.

4. The composition of any preceding claim, wherein the anti-IL-33 antibody comprises a heavy chain variable domain comprising the amino acid sequence of SEQ ID NO: 4, a light chain comprising the amino acid sequence of SEQ ID NO: 8, or a heavy chain variable domain comprising the amino acid sequence of SEQ ID NO: 4 and a light chain comprising the amino acid sequence of SEQ ID NO: 8.

5. The composition of any preceding claim, wherein the anti-IL-33 antibody comprises a heavy chain comprising the amino acid sequence of SEQ ID NO:9, a light chain comprising the amino acid sequence of SEQ ID NO: 10, or a heavy chain comprising the amino acid sequence of SEQ ID NO:9 and a light chain comprising the amino acid sequence of SEQ ID NO:10.

6. The composition of any preceding claim, wherein the anti-IL-33 antibody competes for binding to IL-33 with 33_640087-7B, in an in vitro HTRF competitive binding assay.

7. The composition of any preceding claim, wherein the anti-IL-33 antibody is present at a concentration less than about 200 mg/ml, optionally less than about 180 mg/ml, optionally from about 130 mg/ml to about 170 mg/ml.

8. The composition of any preceding claim, wherein the anti-IL-33 antibody is present at a concentration of 150 mg/ml±10%.

9. The composition of any preceding claim, further comprising a surfactant.

10. The composition of claim 9, wherein the surfactant is polysorbate, optionally wherein the surfactant is polysorbate 20, polysorbate 80 or a mixture thereof.

11. The composition of any one of claims 9 or 10, wherein the surfactant is at a concentration of about 0.005% (w/v) to about 0.05% (w/v).

12. The composition of claim 11, comprising 0.03% (w/v)±0.010% (w/v) surfactant.

13. The composition of any one of claims 10 to 12, wherein the surfactant is polysorbate 80.

14. The composition of any preceding claim, comprising at least about 190 mM arginine, optionally about 190 mM to about 250 mM arginine.

15. The composition of any preceding claim, comprising about 220 mM arginine.

16. The composition of any preceding claim, wherein the arginine is L-arginine hydrochloride.

17. The composition according to any of claims 1 to 13, comprising about 150 mM to about 250 mM lysine, optionally about 170 mM lysine.

18. The composition of any preceding claim, wherein the buffer is histidine, succinate or acetate.

19. The composition of claim 18, wherein the buffer is histidine, optionally wherein the buffer is L-histidine/L-histidine hydrochloride.

20. The composition of any preceding claim, wherein the buffer is at a concentration of about 10 mM to about 30 mM, optionally about 16 mM to about 24 mM, optionally about 17 mM to about 24 mM, optionally about 18 mM to about 24 mM.

21. The composition of either of claims 19 or 20, wherein the buffer is at a concentration of about 19 mM to about 21 mM, optionally 20 mM±10% buffer.

22. The composition of any preceding claim, which is a liquid.

23. The composition of any preceding claim, wherein the pH is less than about pH 6.0, optionally wherein the pH is about pH 5.0 to about pH 6.0.

24. The composition of any preceding claim, wherein the pH is about pH 5.5.

25. The composition of any one of claims 22 to 24, characterized by a reduced viscosity, relative to the viscosity of the anti-IL-33 antibody in a composition comprising 20 mM histidine, 80 mM arginine, 120 mM sucrose, 0.02% (w/v) polysorbate 80 at pH 6.0 and a lower concentration of the anti-IL-33 antibody, for example 50 mg/ml.

26. The composition of any of claims 22 to 25, wherein the viscosity is from about 5 cP to about 20 cP, optionally less than about 10 cP, such as about 9 cP.

27. The composition of any one of claims 22 to 26, characterized by the anti-IL-33 antibody having reduced reversible self-association relative to the reversible self-association of the anti-IL-33 antibody in a composition comprising 20 mM histidine, 80 mM arginine, 120 mM sucrose, 0.02% (w/v) polysorbate 80 at pH 6.0 and a lower concentration of the anti-IL-33 antibody, for example 50 mg/ml.

28. The composition of any preceding claim, comprising a weight % monomer of about 35% to about 50%.

29. The composition of any preceding claim, wherein less than about 5%, optionally less than about 2%, of the antibody aggregates after about 12 months to about 18 months of storage between about 2° C. to about 8° C., as measured by size exclusion chromatography (SEC).

30. A composition comprising:
i. about 130 mg/ml to about 170 mg/mi 33_640087-7B, about 0.03% (w/v)±0.015% polysorbate 80, about 220 mM arginine, and about 16 to about 24 mM histidine buffer, wherein the pH is pH 5.5±0.5;
ii. about 130 mg/ml to about 170 mg/mi 33_640087-7B, about 0.02% (w/v)±0.015% polysorbate 80, about 170 mM lysine, and about 16 to about 24 mM histidine buffer, wherein the pH is pH 5.5±0.5; or
iii. about 130 mg/ml to about 170 mg/mi 33_640087-7B, about 0.02% (w/v)±0.015% polysorbate 80, about 190 mM arginine, and about 16 to about 24 mM histidine buffer, wherein the pH is pH 5.5±0.5

31. The composition of claim 30, comprising about 150 mg/mi 33_640087-7B.

32. The composition of either of claims 30 or 31, comprising about 18 mM to about 22 mM histidine buffer, optionally about 20 mM histidine buffer.

33. The composition of any of claims 30 to 32, which is a liquid.

34. An article of manufacture, comprising the composition of any preceding claim, optionally comprising 0.5 ml to about 5 ml (e.g., 1 ml to about 3 ml) of the composition.

35. A vial comprising the composition of any of claims 1 to 33, optionally comprising about 0.5 ml to about 5 ml (e.g., 1 ml to about 3 ml) of the composition.

36. A method of treating an IL-33-mediated disorder in a subject comprising administering to the subject a therapeutically effective amount of the composition of any one of claims 1 to 33.

37. The method of claim 36, wherein the IL-33-mediated disorder is asthma, atopic dermatitis or chronic obstructive pulmonary disorder, or diabetic kidney disease.

38. A method of making a stable, liquid composition having a viscosity of less than about 10 cP, and comprising greater than about 100 mg/ml of an anti-IL-33 antibody, at least about 170 mM arginine or about 150 mM lysine, a surfactant and a buffer, comprising the steps of:
i. combining in a solution the antibody, arginine and a buffer, to obtain a solution comprising about 100 mg/mL to about 200 mg/mL anti-IL-33 antibody, at least about 170 mM arginine or about 150 mM lysine, and buffer; and
ii. adding a surfactant to the solution to achieve a final surfactant concentration of about 0.03% (w/v)±0.015% (w/v).

39. The method of claim 38, wherein the stable, liquid composition comprises about 220 mM arginine, optionally wherein the arginine is L-arginine hydrochloride.

40. The method of either claim 38 or 39, wherein the surfactant is polysorbate 80 and the buffer is made from histidine.

41. The method of any of claims 38 to 40, the method results in a stable, liquid formulation having a pH of from about pH 5 to about pH 6, optionally about pH 5.5.

42. The method of any one of claims 38 to 41, wherein the anti-IL-33 antibody is as defined in any of claims 1 to 5.

43. The method of any one of claims 38 to 42, wherein cP is measured at 23° C.
